(19) United States
(12) Patent Application Publication
(10) Pub. No.: US 2009/0048675 A1
(43) Pub. Date: Feb. 19, 2009

(54) Spinal Fusion Implants with Selectively Applied Bone Growth Promoting Agent

(76) Inventors: Mohit K. Bhatnagar, Potomac, MD (US); Jack Y. Yeh, North Potomac, MD (US)

Correspondence Address: PLUMSEA LAW GROUP, LLC, 10411 MOTOR CITY DRIVE, SUITE 320, BETHESDA, MD, 20817, US

(21) Appl. No.: 11/840,707

(22) Filed: Aug. 17, 2007

(51) Int. Cl. A61B 17/58; A61F 2/02; A61F 2/44; A61B 17/70; A61B 17/56

(52) U.S. Cl. 623/17.16; 623/23.76; 606/280; 606/286; 606/76

(57) ABSTRACT

A spinal fusion device including a selectively applied bone growth promoting agent is disclosed. In particular, a bone growth promoting agent is selectively applied to spinal implants, spinal plugs, spinal wedges and other implantable devices.

### BACKGROUND OF THE INVENTION

1. Field of the Invention

The present invention relates generally to implantable prostheses and in particular to a spinal fusion device including a selectively applied bone growth promoting agent.

2. Description of Related Art

Spinal fusion implants have been previously proposed. In some cases, spinal fusion devices are embedded between adjacent vertebrae, partially or fully replacing the tissue disposed between the vertebrae.

One type of spinal fusion device is disclosed in Michelson (U.S. Pat. No. 6,264,656), the entirety of which is incorporated by reference. The threaded spinal implant of Michelson is inserted between two adjacent vertebrae and is designed to fuse those vertebrae in the spine.

Brantigan (U.S. Pat. No. 4,834,757) discloses plugs, used as spinal fusion devices, the entirety of which is incorporated by reference. The plugs are rectangular with tapered front ends and tool receiving rear ends. Generally, the plugs may be used in a similar manner to the spinal implants of Michelson. As with the spinal implants, the plugs may be inserted between adjacent vertebrae. The plugs may include nubs that behave like teeth, countering any tendency for the plugs to slip between the vertebrae.

While the related art teaches various forms of spinal fusion devices, there are many shortcomings. Related art prostheses lack selectively applied bone growth promoting treatments. The prior art does not teach the selective application of the variety of known bone growth promoting treatments. There is therefore a need in the art for prostheses that incorporate selectively applied bone growth promoting treatments.

### SUMMARY OF THE INVENTION

A spinal fusion device including a selectively applied bone growth promoting agent is disclosed. In one aspect, the invention provides a spinal fusion device, comprising: a spinal implant configured for insertion between two vertebrae; the spinal implant including a first portion and a second portion along an outer surface; a bone growth promoting agent; and where the bone growth promoting agent is selectively applied to the first portion of the outer surface.

In another aspect, the bone growth promoting agent is selectively applied to an inner surface of the spinal implant.

In another aspect, the spinal implant includes a plurality of holes.

In another aspect, the plurality of holes are disposed on an outer surface of the spinal implant.

In another aspect, the plurality of holes includes small holes and large holes.

In another aspect, the bone growth promoting agent is selectively applied to at least one of the plurality of holes.

In another aspect, the spinal implant has a solid portion.

In another aspect, the spinal implant has a hollow portion.

In another aspect, the spinal implant has a latticed portion.

In another aspect, the invention provides a spinal fusion device, comprising: a spinal implant configured for insertion between two vertebrae; the spinal implant including threading; the threading including threading peaks and threading valleys; a bone growth promoting agent; and where the bone growth promoting agent is selectively applied to the threading peaks.

In another aspect, the threading peaks include an upper portion, a middle portion and a lower portion.

In another aspect, the bone growth promoting agent is selectively applied to a member of the group consisting essentially of the upper portion, the lower portion, the middle portion and the threading valleys.

In another aspect, the spinal implant includes a plurality of holes.

In another aspect, at least one of the plurality of holes penetrates from an outer surface of the spinal implant to an inner surface associated with a hollow central core.

In another aspect, at least one of the plurality of holes has a bottom.

In another aspect, the invention provides a spinal fusion device, comprising: a spinal plug configured for insertion between two vertebrae; the spinal plug including a first portion and a second portion along an outer surface; a bone growth promoting agent; and where the bone growth promoting agent is selectively applied to the first portion of the outer surface.

In another aspect, the bone growth promoting agent is selectively applied to a portion of an inner surface of the spinal plug.

In another aspect, the spinal plug has a solid portion.

In another aspect, the spinal plug has a hollow portion.

In another aspect, the spinal plug has a latticed portion.

In another aspect, the spinal plug includes a plurality of holes.

In another aspect, the bone growth promoting agent is selectively applied to at least one of the holes.

In another aspect, the plurality of holes are disposed on a top side and a bottom side of the spinal plug.

In another aspect, the plurality of holes includes small holes and large holes.

In another aspect, the invention provides a spinal fusion device, comprising: a spinal wedge configured for insertion between two vertebrae; the spinal wedge including a first portion and a second portion along an outer surface; a bone growth promoting agent; and where the bone growth promoting agent is selectively applied to the first portion of the outer surface.

In another aspect, the spinal wedge includes a hollow portion.

In another aspect, the spinal wedge includes a solid portion.

In another aspect, the spinal wedge includes a latticed portion.

In another aspect, the implantable device includes a plurality of holes.

In another aspect, the plurality of holes are disposed on a top side and a bottom side of the spinal wedge.

In another aspect, the plurality of holes includes small holes and large holes.

In another aspect, the bone growth promoting agent is selectively applied to at least one of the plurality of holes.

In another aspect, the invention provides a spinal fusion device, comprising: an implantable device configured for insertion between two vertebrae; the implantable device including a first portion and a second portion along an outer surface; a bone growth promoting agent; and where the bone growth promoting agent is selectively applied to the first portion of the outer surface.

In another aspect, the implantable device includes teeth.

In another aspect, the implantable device includes a sloped top side.

In another aspect, the implantable device includes a sloped bottom side.

In another aspect, the implantable device includes a plurality of holes.

In another aspect, the plurality of holes are disposed on the sloped top side and the sloped bottom side.

In another aspect, the plurality of holes includes small holes and large holes.

In another aspect, the bone growth promoting agent is selectively applied to at least one of the plurality of holes.

In another aspect, the invention provides a spinal fusion device, comprising: an implantable device configured for insertion between two vertebrae; the implantable device including a first portion and a second portion; a bone growth promoting agent; a lattice structure disposed within the implantable device; and where the bone growth promoting agent is selectively applied to the first portion.

In another aspect, the first portion includes a portion of a shell of the implantable device.

In another aspect, the first portion also includes a portion of the lattice structure, wherein the bone growth promoting agent applied to both the shell and the lattice structure encourages bone growth into the lattice structure and bone integration with the lattice structure.

In another aspect, the first portion includes a portion of the lattice structure.

In another aspect, the lattice structure is removable from the spinal fusion device.

In another aspect, the invention provides a spinal fusion device, comprising: an implantable device including a surface associated with a vertebra; the surface including a hole; and where a bone growth promoting agent is selectively applied to a portion of the hole.

In another aspect, the hole extends through the surface.

In another aspect, the hole includes a bottom.

In another aspect, the hole is microscopic.

In another aspect, the hole is macroscopic.

In another aspect, the invention provides a bone fusion device, comprising: a body portion; a first inserting portion extending from the body portion; a second inserting portion extending from the body portion; wherein the first inserting portion engages a first bone and wherein the second inserting portion engages a second bone; and wherein a bone growth promoting agent is selectively applied to a portion of the bone fusion device.

In another aspect, the bone fusion device includes at least one hole, and wherein the hole is microscopic.

In another aspect, the bone fusion device includes at least one hole, and wherein the hole is macroscopic.

In another aspect, the first bone is a vertebrae and wherein the second bone is an adjacent vertebrae.

In another aspect, the bone fusion device includes at least two inserting portions.

In another aspect, the bone fusion device includes at least four inserting portions.

Other systems, methods, features and advantages of the invention will be, or will become, apparent to one of ordinary skill in the art upon examination of the following figures and detailed description. It is intended that all such additional systems, methods, features and advantages be included within this description and this summary, be within the scope of the invention, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views.

FIG. 1 is an isometric view of a preferred embodiment of a rod;

FIG. 2 is a cross sectional view of a preferred embodiment of a rod;

FIG. 3 is a cross sectional view of a preferred embodiment of a rod;

FIG. 4 is a plan view of a preferred embodiment of a sheet material;

FIG. 5 is an isometric view of a preferred embodiment of a sheet material being applied to a rod;

FIG. 6 is an isometric view of a preferred embodiment of a rod and a sleeve;

FIG. 7 is an isometric view of a preferred embodiment of a rod with a bone growth promoting agent;

FIG. 8 is an isometric view of a preferred embodiment of a rod with a bone growth promoting agent applied along a single portion;

FIG. 9 is an isometric view of a preferred embodiment of a rod with a bone growth promoting agent applied along several portions;

FIG. 10 is an isometric view of a preferred embodiment of a rod with a bone growth promoting agent applied as a striped pattern;

FIG. 11 is an isometric view of a preferred embodiment of a rod with a bone growth promoting agent applied as a spotted pattern;

FIG. 12 is an isometric view of a preferred embodiment of a rod with a bone growth promoting agent applied as a geometric pattern;

FIG. 13 is an isometric view of a preferred embodiment of a rod with a bone growth promoting agent applied as a spiral pattern;

FIG. 14 is an isometric view of a preferred embodiment of a rod with a bone growth promoting agent applied as various patterns;

FIG. 15 is an isometric view of a preferred embodiment of a rod with a bone growth promoting agent applied as various patterns;

FIG. 16 is an isometric view of a preferred embodiment of a rod with a modified surface texture;

FIG. 17 is a side view of a preferred embodiment of a microscopic surface texture;

FIG. 18 is a side view of a preferred embodiment of a microscopic surface texture;

FIG. 19 is a side view of a preferred embodiment of a microscopic surface texture;

FIG. 20 is a top down view of a preferred embodiment of a three dimensional surface texture;

FIG. 21 is a top down view of a preferred embodiment of a three dimensional surface texture;

FIG. 22 is a top down view of a preferred embodiment of a three dimensional surface texture;

FIG. 23 is an isometric view of a preferred embodiment of a rod with various bone growth promoting agents;

FIG. 24 is an isometric view of a preferred embodiment of a solid rod;

FIG. 25 is an isometric view of a preferred embodiment of a hollow rod;

FIG. 26 is an isometric view of a preferred embodiment of a solid rod with holes;

FIG. 27 is an isometric view of a preferred embodiment of a hollow rod with holes;

FIG. 28 is a schematic cross sectional view of a preferred embodiment of a hollow rod with holes;

FIG. 29 is a schematic cross sectional view of a preferred embodiment of a rod inserted into bone;

FIG. 30 is a schematic cross sectional view of a preferred embodiment bone growing into a rod;

FIG. 31 is a cross sectional view of a preferred embodiment of an implantable prosthesis system;

FIG. 32 is an isometric view of a preferred embodiment of a fracture plate configured to attach to a bone;

FIG. 33 is an isometric view of a preferred embodiment of a fracture plate with a bone growth promoting agent;

FIG. 34 is an isometric view of a preferred embodiment of a fracture plate with a bone growth promoting agent;

FIG. 35 is an isometric view of a preferred embodiment of a fracture plate with a bone growth promoting agent;

FIG. 36 is an isometric view of a preferred embodiment of a fracture plate with a bone growth promoting agent;

FIG. 37 is an isometric view of a preferred embodiment of a fracture plate with a bone growth promoting agent;

FIG. 38 is an isometric view of a preferred embodiment of a fracture plate with a bone growth promoting agent;

FIG. 39 is an isometric view of a preferred embodiment of a fracture plate with a bone growth promoting agent;

FIG. 40 is an isometric view of a preferred embodiment of a fracture plate with a bone growth promoting agent;

FIG. 41 is an isometric view of a preferred embodiment of a fracture plate with a bone growth promoting agent;

FIG. 42 is an isometric view of a preferred embodiment of a liner system;

FIG. 43 is a side cross sectional view of a preferred embodiment of a fracture plate contacting a bone;

FIG. 44 is a side cross sectional view of a preferred embodiment of a fracture plate with bony fusion;

FIG. 45 is a schematic cross section of a preferred embodiment of the threading of a screw;

FIG. 46 is a schematic cross section of a preferred embodiment of the threading of a screw;

FIG. 47 is a schematic cross section of a preferred embodiment of the threading of a screw;

FIG. 48 is a schematic cross section of a preferred embodiment of the threading of a screw;

FIG. 49 is a schematic cross section of a preferred embodiment of the threading of a screw;

FIG. 50 is a schematic cross section of a preferred embodiment of the threading of a screw;

FIG. 51 is a side view of a preferred embodiment of a screw;

FIG. 52 is a side view of a preferred embodiment of a screw;

FIG. 53 is a side view of a preferred embodiment of a screw;

FIG. 54 is a side view of a preferred embodiment of a screw;

FIG. 55 is a close up cross sectional view of a screw with a hollow boring tip;

FIG. 56 is a close up cross sectional view of a screw with a solid boring tip;

FIG. 57 is a schematic cross section of a preferred embodiment of a screw inserted into bone;

FIG. 58 is a schematic cross section of a preferred embodiment of bone growing into a hollow central core of a screw;

FIG. 59 is a preferred embodiment of a barrel shaped spinal implant implanted between two vertebrae;

FIG. 60 is an isometric view of a preferred embodiment of the lower half of a barrel shaped spinal implant;

FIG. 61 is a side view of a preferred embodiment of a barrel shaped spinal implant with threading including a selectively applied bone growth promoting agent;

FIG. 62 is a side view of a preferred embodiment of a barrel shaped spinal implant with threading including a selectively applied bone growth promoting agent;

FIG. 63 is a side view of a preferred embodiment of a barrel shaped spinal implant with threading including a selectively applied bone growth promoting agent;

FIG. 64 is a preferred embodiment of a conically shaped spinal implant implanted between two vertebrae;

FIG. 65 is an isometric view of a preferred embodiment of the lower half of a conically shaped spinal implant;

FIG. 66 is a side view of a preferred embodiment of a conically shaped spinal implant with threading including a selectively applied bone growth promoting agent;

FIG. 67 is a side view of a preferred embodiment of a conically shaped spinal implant with threading including a selectively applied bone growth promoting agent;

FIG. 68 is a side view of a preferred embodiment of a conically shaped spinal implant with threading including a selectively applied bone growth promoting agent;

FIG. 69 is a side view of a preferred embodiment of a self tapping spinal implant;

FIG. 70 is a cross sectional view of a preferred embodiment of a barrel shaped spinal implant implanted between two vertebrae;

FIG. 71 is a cross sectional view of a preferred embodiment of a barrel shaped spinal implant implanted between two vertebrae;

FIG. 72 is a cross sectional view of a preferred embodiment of a conically shaped spinal implant implanted between two vertebrae;

FIG. 73 is a cross sectional view of a preferred embodiment of a conically shaped spinal implant implanted between two vertebrae;

FIG. 74 is a side view of a preferred embodiment of a spinal wedge implanted between two vertebrae;

FIG. 75 is an isometric view of a preferred embodiment of a spinal wedge;

FIG. 76 is an isometric view of a preferred embodiment of a spinal plug;

FIG. 77 is an isometric view of a preferred embodiment of an implantable device;

FIG. 78 is a cross sectional view of a preferred embodiment of a spinal implant implanted between two vertebrae;

FIG. 79 is a cross sectional view of a preferred embodiment of a spinal implant implanted between two vertebrae;

FIG. 80 is a side view of a preferred embodiment of a spinal implant with two screws;

FIG. 81 is a side view of a preferred embodiment of a spinal implant with keystones;

FIG. 82 is a cross sectional view of a preferred embodiment of a spinal implant;

FIG. 83 is a side view of a preferred embodiment of a spinal implant with a double pitch;

FIG. 84 is an isometric view of a preferred embodiment of a lower half of a barrel shaped spinal implant with an inner lattice;

FIG. 85 is an isometric view of a preferred embodiment of a lower half of a barred shaped spinal implant with an inner lattice;

FIG. 86 is an isometric view of a preferred embodiment of a bone staple inserted into two adjacent vertebrae;

FIG. 87 is a schematic diagram of a preferred embodiment of a bone staple inserted into two adjacent vertebrae with a selectively applied bone growth promoting agent;

FIG. 88 is a schematic diagram view of a preferred embodiment of a bone staple inserted into two adjacent vertebrae with bone growth;

FIG. 89 is a rear isometric view of a preferred embodiment of two bone staples inserted into two adjacent vertebrae;

FIG. 90 is a rear isometric view of a preferred embodiment of a wide bone staple configured for insertion into two adjacent vertebrae with a selectively applied bone growth promoting agent;

FIG. 91 is a rear isometric view of a preferred embodiment of a wide bone staple inserted into two adjacent vertebrae; and

FIG. 92 is a rear isometric view of a preferred embodiment of a wide bone staple inserted into two adjacent vertebrae with bone growth.

### DETAILED DESCRIPTION OF THE PREFERRED EMBODIMENTS

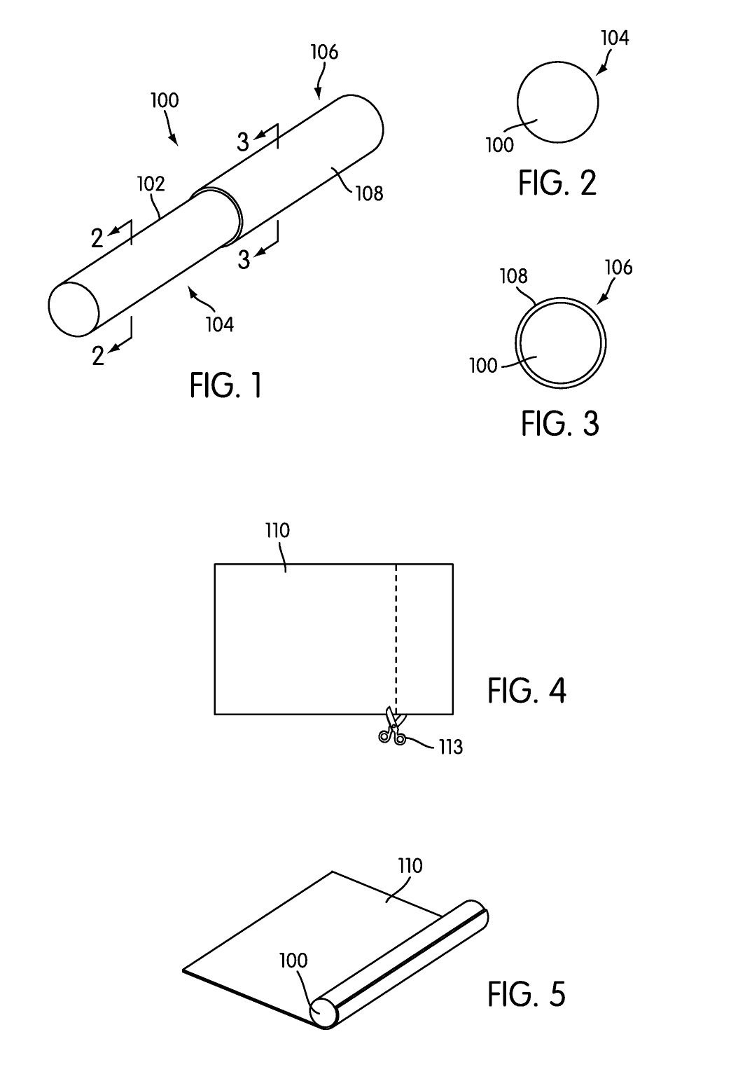

FIG. 1 is a preferred embodiment of an implantable prosthesis in the form of rod 100. For clarity, the following detailed description discusses a preferred embodiment, however, it should be kept in mind that the present invention could also take the form of any other kind of implantable prosthesis including, for example, screws, fracture plates, cages, connectors, wires, cables, clamps, staples, anchors or any other kind of prosthesis.

Often, an implantable prosthesis may include a provision for promoting bone growth. Generally, throughout this specification and the claims, such a provision will be referred to as a bone growth promoting agent. Bone growth promoting agents may be divided into two categories. The first category includes any provision that uses additive components to the prosthesis itself. The second category includes any provision that modifies the surface structure of the prosthesis, which is often metallic.

The first category may include provisions that are freeze dried onto a surface or adhered to the metal through the use of linker molecules or a binder. Examples of the first category that may be applied through these techniques include, but are not limited to, bone morphogenetic proteins (BMPs), such as BMP-1, BMP-2, BMP-4, BMP-6, and BMP-7. These are hormones that convert stem cells into bone forming cells. Further examples include recombinant human BMPs (rhBMPs), such as rhBMP-2, rhBMP-4, and rhBMP-7. Still further examples include platelet derived growth factor (PDGF), fibroblast growth factor (FGF), collagen, BMP mimetic peptides, as well as RGD peptides. Generally, combinations of these chemicals may also be used. These chemicals can be applied using a sponge, matrix or gel.

Some chemicals from the first category may also be applied to an implantable prosthesis through the use of a plasma spray or electrochemical techniques. Examples of these chemicals include, but are not limited to, hydroxyapatite, beta tri-calcium phosphate, calcium sulfate, calcium carbonate, as well as other chemicals.

Provisions from the second category generally modify the surface structure of the prosthesis. In some cases, the surface structure is roughened or provided with irregularities. Generally, this roughened structure may be accomplished through the use of acid etching, bead or grit blasting, sputter coating with titanium, sintering beads of titanium or cobalt chrome onto the implant surface, as well as other methods. This can result in a prosthesis with a surface roughness with about 3-5 microns of roughness peak to valley. However, in some embodiments, the surface roughness may be less than 3-5 microns peak to valley, and in other embodiments, the surface roughness may be greater than 3-5 microns peak to valley. In some exemplary embodiments, the prosthesis can be made of commercially pure titanium or a titanium alloy (such as Ti6Al4V) with about 3-5 microns of roughness peak to valley.

It should be understood that the provisions listed here are not meant to be an exhaustive list of possible bone growth promoting agents. The term bone growth promoting agent, as used in this specification and claims, is intended to include any method of modifying an implantable prosthesis that stimulates bone growth either directly or indirectly.

Rod 100 preferably includes outer surface 102. In some embodiments, outer surface 102 preferably includes first portion 104 and second portion 106. In this embodiment, coating 108 has been applied to second portion 106 of outer surface 102. In a preferred embodiment, coating 108 includes a bone growth promoting agent of some kind.

Referring to FIGS. 2-3, cross sections of first portion 104 and second portion 106 preferably differ. In particular, second portion 106 preferably includes coating 108. In this embodiment, coating 108 preferably has some thickness. In other embodiments, the thickness of coating 108 may be varied.

As previously mentioned, bone growth promoting agents may be applied in a variety of ways. In some embodiments, bone growth promoting agents may be applied to a mesh or fabric material that may be independently manufactured from the implantable prosthesis. In this manner, the fabric or mesh material, which includes the bone growth promoting agent, may be applied to the implantable prosthesis at any time prior to surgery, during surgery or even after implantation. In addition to mesh or a fabric material, the sheet can be any kind of bio-compatible material that includes a metallic foil, a plastic sheet or a biological matrix. The metal can be titanium, stainless steel, cobalt chrome or any other type of bio-compatible metal or matrix.

Referring to FIGS. 4-5, sheet material 110 may be constructed to include a bone growth promoting agent. In some embodiments, sheet material 110 may be any material that may be configured to include a bone growth promoting agent, and that is flexible enough to wrap around an implantable prosthesis. In a preferred embodiment, sheet material 110 may be a mesh or continuous fabric. In this embodiment, scissors 113 may be used to cut sheet material 110 to a preconfigured size, which can be any desired size.

Once sheet material 110 has been cut to an appropriate size, it may be applied to rod 100. Generally, sheet material 110 may be rolled over rod 100. In some embodiments, sheet material 110 may be attached to rod 100 through an adhesive. It is also possible to attach sheet material 110 to rod 100 by using mechanical provisions, including hooks, microscopic hooks, temperature difference, interference fit or a Morris taper. It is also possible to attach sheet material 110 to rod 100 using magnetic features. In a preferred embodiment, sheet material 110 may be preconfigured to include an adhesive for attaching to rod 100.

In some embodiments, a sheet material may be preconfigured as a sleeve or any desired shape. Preferably, the sleeve may be configured so that a rod or another type of prosthesis may be inserted into the sleeve, without the need to wrap the sheet material around the prosthesis. The sleeve can come in a variety of sizes and shapes. Like the sheet material, the sleeve material may be constructed of a continuous or mesh fabric, collagen, or biologic matrix, metallic foil or plastic sheet.

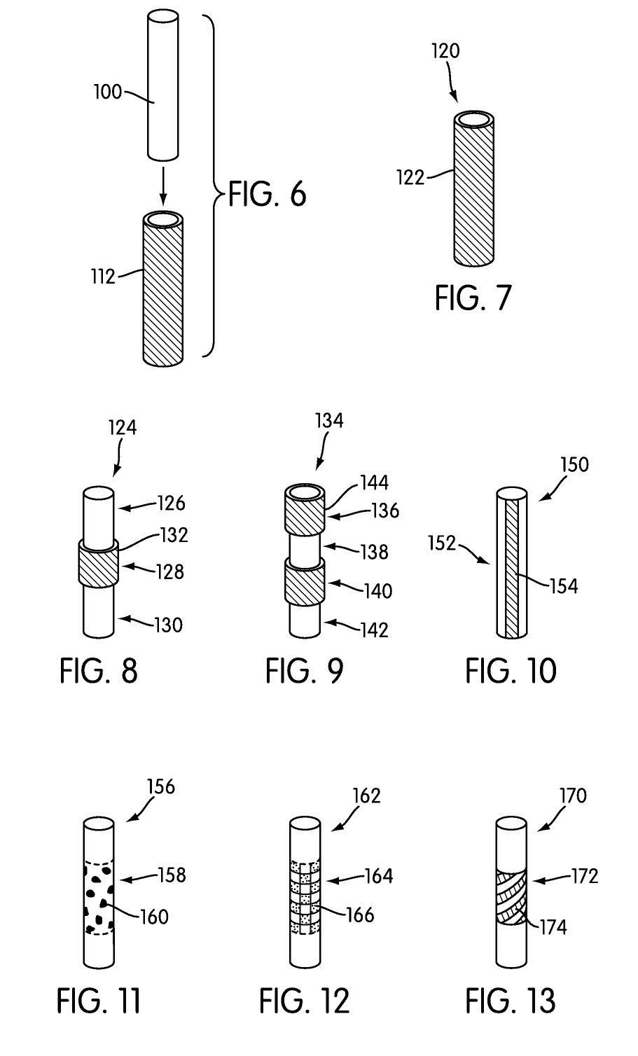

Referring to FIG. 6, sleeve material 112 may be constructed to include a bone growth promoting agent. Preferably, sleeve material 112 may be configured to receive all or a portion of a rod 100. Generally, sleeve material 112 may be configured to receive all or a portion of an implantable prosthesis. In this manner, a bone growth promoting agent may be applied via sleeve material 112 by simply inserting the prosthesis into sleeve material 112. This configuration allows a bone growth promoting agent to be applied to a rod in an efficient manner.

Preferably, sheet material 110 and sleeve material 112 may be applied to multiple types of implantable prosthesis, including, but not limited to screws, fracture plates, cages, connectors, wires, cables, clamps, staples, anchors or any other kind of prosthesis. In some embodiments, sheet material 110 may be cut to a size configured to cover all or a portion of an implantable prosthesis. Additionally, sleeve material 112 may be constructed in a manner that allows all or a portion of an implantable prosthesis to be inserted into sleeve material 112.

Preferably, a rod intended to be used as a prosthesis includes provisions for selectively applying a bone growth promoting agent to various portions of the rod. In other words, a bone growth promoting agent need not be applied to the entirety of the rod. Instead, the bone growth promoting agent may be applied to a single portion of the rod. In some embodiments, the bone growth promoting agent may be applied to multiple, but not all, portions of the rod. Additionally, the bone growth promoting agent may be applied differently along different portions of the rod. In this manner, the rod may be used to differentially stimulate bone growth along various portions of the adjacent bone to simulate fusion, healing, stabilization and/or incorporation. This may be useful in cases where some, but not all, portions of the bone are damaged.

Referring to FIGS. 7-9, several embodiments of a rod may include a bone growth promoting agent that has been applied along various portions. For the purposes of illustration, the thicknesses of the portions including a bone growth promoting agent have been exaggerated. Generally, these thicknesses may vary. Some bone growth promoting agents may be applied to the surface of a rod, or other prosthesis, and have no visible thickness.

In some embodiments, the bone growth promoting agent may be applied to the entirety of the rod. Rod 120 preferably includes bone growth promoting agent 122 along the entirety of the length of rod 120. Bone growth promoting agent 122 may be any of the possible provisions discussed previously for applying a bone growth promoting agent to an implantable prosthesis. With this configuration, rod 120 may help to stimulate bone growth along the entirety its length, following the implantation of rod 120.

In other embodiments, a rod may include three portions, with only one portion including a bone growth promoting agent. Rod 124 preferably includes first portion 126, second portion 128, and third portion 130. In a preferred embodiment, second portion 128 includes bone growth promoting agent 132. With this configuration, rod 124 may help to stimulate bone growth along a portion of the bone adjacent to second portion 128, following the implantation of rod 124.

In another embodiment, a rod may include four portions, with alternating portions including a bone growth promoting agent. Preferably, rod 134 may include first portion 136, second portion 138, third portion 140, and fourth portion 142. In some embodiments, only first portion 136 and third portion 140 include bone growth promoting agent 144. With this configuration, rod 134 may help to stimulate bone growth along portions of the bone adjacent to first portion 136 and third portion 140, following the implantation of rod 134. In other embodiments, more or less than four portions may be provided.

In the previous embodiments, along portions where a bone growth promoting agent has been applied, it has been preferably applied uniformly throughout the portion. In some embodiments, however, a bone growth promoting agent may be applied in particular patterns throughout a portion. Depending on the circumstances, different types of patterns may be used to promote bone growth.

Examples of some patterns include stripes, spots, helical or spiral, geometric patterns, or combinations incorporating one or more of these basic pattern elements. The term geometric pattern refers to any polygonal pattern including square (shown in the Figures), rectangular, polygon, honeycomb, repeating, non-repeating, regular, irregular, as well as other types of patterns. A striped pattern includes thin lines of bone growth promoting agent that are disposed along a particular portion. In this arrangement, there is no bone growth promoting agent between the stripes. A spotted pattern may include small spots of the bone growth promoting agent. In a similar manner, a geometric pattern may include alternating shapes of a bone growth promoting agent. Various patterns may be used depending on the way in which the user wants to induce bone growth along or adjacent to the prosthetic.

FIGS. 10-13 illustrate various patterns of bone growth promoting agents applied to rods. Rod 150 preferably includes first portion 152. In some embodiments, first portion 152 may include bone growth promoting agent 154. In a preferred embodiment, bone growth promoting agent 154 may be disposed in a striped pattern as shown in FIG. 10. This striped pattern may include one or more stripes. Generally, the thickness and/or density of these stripes may be varied. Additionally, their orientation may also be varied. The shape, density and/or distribution of the bone growth promoting agent will allow for selectively tailored bone growth or fusion.

In a second embodiment, rod 156 preferably includes first portion 158. In some embodiments, first portion 158 may include bone growth promoting agent 160. In a preferred embodiment, bone growth promoting agent 160 may be disposed in spots along first portion 158. Generally, the shape and/or density of these spots may be varied.

In a third embodiment, rod 162 preferably includes first portion 164. In some embodiments, first portion 164 may include bone growth promoting agent 166. In a preferred embodiment, bone growth promoting agent 166 may be disposed in a geometric pattern along first portion 164. Generally, the size of the squares comprising this geometric pattern may be varied.

In a fourth embodiment, rod 170 preferably includes first portion 172. In some embodiments, first portion 172 may include bone growth promoting agent 174. In a preferred embodiment, bone growth promoting agent 174 may be disposed in a spiral or helical pattern along first portion 172. Generally, the thickness and spacing of this spiral pattern may be varied.

The patterns disclosed here are not intended to be exhaustive, but only illustrative of the various types of patterns that may be included in portions where a bone growth promoting agent is applied to a rod or other implantable prosthesis. Generally, any type of pattern may be used. Additionally, within the same portion, multiple patterns may be superimposed.

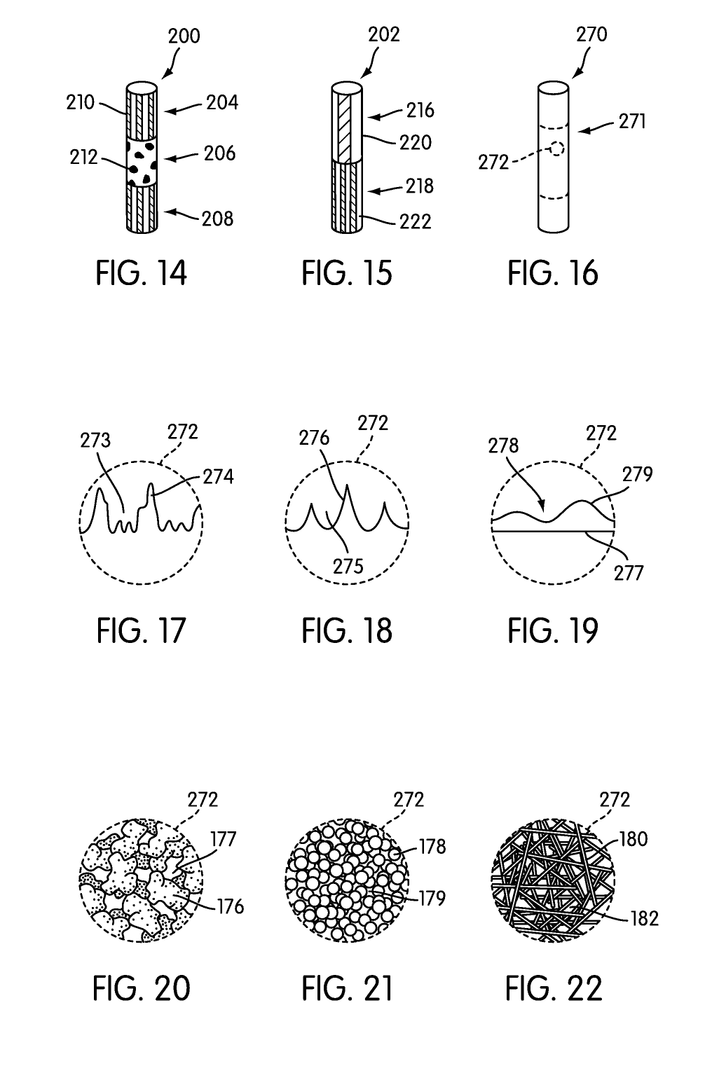

Generally, various patterns of bone growth promoting agents may be selectively applied to multiple portions of a rod or other implantable prostheses. FIGS. 14-15 are a preferred embodiment of first rod 200 and second rod 202. In some embodiments, first rod 200 includes first portion 204, second portion 206, and third portion 208. In some embodiments, a distinct pattern of a bone growth promoting agent may be selectively applied to each of the portions 204, 206, and 208. In a preferred embodiment, first portion 204 and third portion 208 may include bone growth promoting agent 210 arranged as stripes. Likewise, second portion 206 may include bone growth promoting agent 212 arranged as spots.

Preferably, second rod 202 includes first portion 216 and second portion 218. In some embodiments, both first portion 216 and second portion 218 include the same pattern of a bone growth promoting agent. In some embodiments, both portions 216 and 218 include a bone growth promoting agent arranged as stripes. In some embodiments, first portion 216 includes first striped pattern 220 of a bone growth promoting agent, while second portion 218 includes second striped pattern 222 of a bone growth promoting agent. In a preferred embodiment, the density of first striped pattern 220 is lower than the density of second striped pattern 222. First striped pattern 220 can have different a orientation and can be angled with respect to second striped pattern 222.

Referring to FIGS. 16-22, bone growth promoting agents may also be selectively applied to various portions of a rod by modification of the surface properties. Preferably, rod 270 includes first portion 271. In some embodiments, first portion 271 may include a bone growth promoting agent in the form of a textured surface. The structure of this surface may be seen in a close up of patch 272.

In some embodiments, first portion 271 may include a textured surface due to acid etching of titanium. In this case, a side view of patch 272, when viewed at the microscopic level, may include jagged peaks 274 and jagged valleys 273. In another embodiment, first portion 271 may include a textured surface due to grit blasting the titanium. In this case, a side view of patch 272, when viewed at a microscopic level, may include sharp peaks 276 and smooth valleys 275. Finally, in an embodiment where plasma spraying is used to texture the surface of portion 271, a side view of patch 272 may include rounded peaks 279, rounded valleys 278, and under surface 277.

Referring to FIGS. 20-22, some rods may be configured so that the surface includes various three dimensional structures. In some embodiments, first surface 272 may include an irregular three dimensional surface. FIG. 20 shows an embodiment including an irregular porous titanium construct, including irregular structures 176 and first pores 177. In a preferred embodiment, the sizes of first pores 177 may be between 100 and 600 microns. In another embodiment, first surface 272 may include a regular three dimensional surface. FIG. 21 shows an embodiment including a regular ball bearing type structure made of titanium, including ball bearing-like structures 178 and second pores 179. Second pores 179 may also have a size between 100 and 600 microns. In another embodiment, shown in FIG. 22, first surface 272 may include a fibrous three dimensional surface. In this embodiment, the fibrous surface includes fibrous structures 180 and third pores 182. Using these various types of three dimensional structures on the surface of rod 270 allows for an increased surface area for new bone growth, as opposed to traditional surface treatment methods. In particular, the height or thickness of these various surface treatments may be large when compared with traditional surface treatments.

Other surface treatments that can be used include micro-porous coatings. Additionally, any and all coatings, treatments or patterns can be used that promote bone growth or allow for bone growth to the prosthesis and effectively lock the prosthesis to the bone. In some embodiments, these surface treatments can provide the surface of the prosthesis with a roughness of about 3-5 microns, peak to valley, or a pore size of about 1-850 microns as previously discussed. The pore size can be increased if desired. However, in other embodiments, the peak to valley roughness will be greater than 3-5 microns, and in other embodiments, the peak to valley roughness may be less than 3-5 microns, depending on the application. In some cases, these surface treatments will be invisible to the naked eye.

The specific surface treatment feature or combination of features can be selected based on: biology, location, bony region (metaphyseal or cortical bone; weight bearing or non-weight bearing, for example) cost, strength of the implant or prosthesis, geometry or size of the implant or prosthesis and manufacturing feasibility, among other criteria or factors that may be considered.

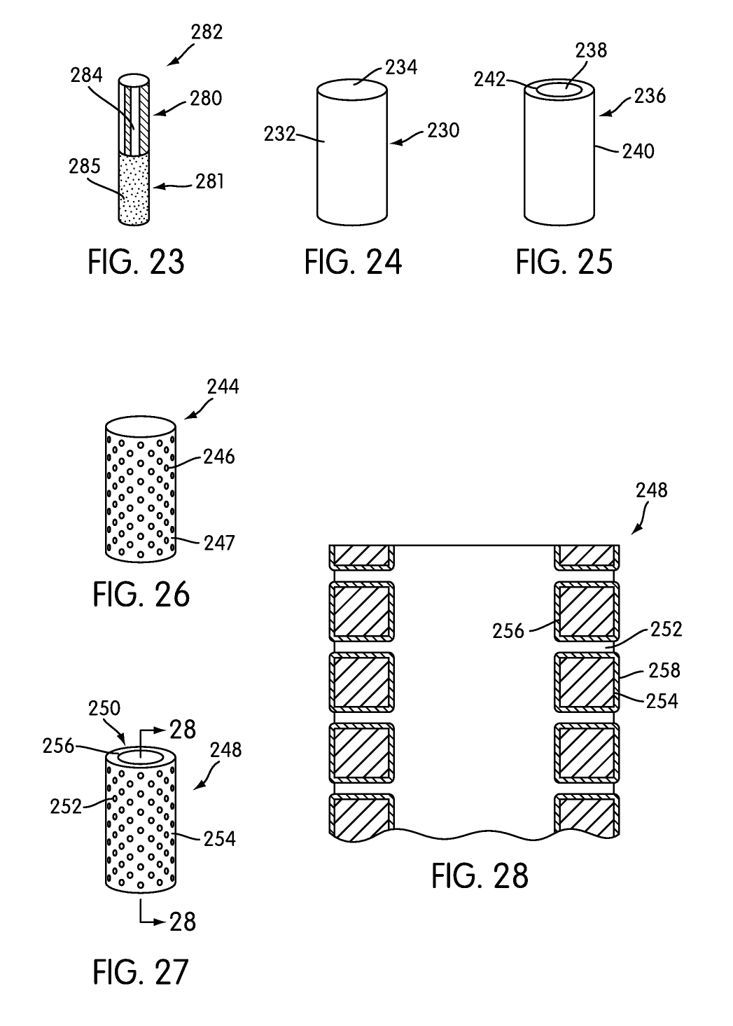

In some embodiments, a rod may include a chemical bone growth promoting agent along one portion and a modified surface bone growth promoting agent along a second portion. In a preferred embodiment, shown in FIG. 23, rod 282 may include first region 280 and second region 281. In some embodiments, each of the regions 280 and 281 may include a different bone growth promoting agent. In a preferred embodiment, first region 280 may include striped pattern 284 of a chemical bone growth promoting agent. Also, second region 281 may include acid etched surface 285, another type of bone growth promoting agent. For the purposes of illustration, acid etched surface 285 is shown here with some shading, but generally, textured surfaces may be invisible to the naked eye.

Generally, some rods include provisions for modifying the structure of the rod. These modifications may include a hollowing out of the core of the rod. Additionally, these modifications may include the addition of holes that may be disposed along the outer surface of the rod and penetrate into the core of the rod.

Referring to FIGS. 24-27, rods may be configured solid, hollow, and with or without holes. If the rod includes holes, the holes can be any desired size and shape. Also, the distribution pattern of the holes may be varied. In one embodiment, a section of rod 230 may be solid. Rod 230 may include outer surface 232. In a preferred embodiment, core 234 of rod 230 may be solid. In a second embodiment, a section of rod 236 may include hollow central core 238. Preferably, rod 236 includes outer surface 240. In a preferred embodiment, rod 236 may also include inner surface 242 of hollow central core 238.

Preferably, a third embodiment of a section of rod 244 may include holes 246. Holes 246 are preferably disposed along the entirety of rod 244 along outer surface 247. Holes 246 may also be disposed along a single portion of rod 244 in other embodiments. Generally, holes 246 may be any depth, any shape, angle, and have any size circumference. Similarly, the density of holes 246 may be varied in other embodiments. In some embodiments, a combination of holes having different sizes, shapes, angles or densities may be used.

A fourth embodiment of a section of rod 248 may preferably include hollow central core 250 as well as holes 252. Holes 252 are preferably disposed along the entirety of rod 248. Generally, holes 252 may be any depth, any shape, angle, and have any size circumference. Similarly, the density of holes 252 may be varied in other embodiments. In some embodiments, a combination of holes having different sizes, shapes, angles or densities may be used. Holes 252 may or may not penetrate through to hollow central core 250. In a preferred embodiment, holes 252 are disposed between outer surface 254 and inner surface 256 of hollow central core 250. In this manner, holes 252 preferably allow fluid communication between hollow central core 250 and outer surface 254, which allows bony ingrowth to occur into the interstices of rod 248.

Preferably, an implantable prosthesis system may include provisions for fusing the prosthesis to the bone. In some embodiments, a rod may be configured to be fused to a bone once it has been implanted. In particular, the rod may include provisions that allow the bone to penetrate through the outer surface and grow along an inner surface of a hollow core or into the holes themselves, and into the bone growth promoting agent of the prosthesis.

In some embodiments, outer surface 254 may include bone growth promoting agent 258, seen in FIG. 28, a cross sectional view of rod 248. In some embodiments, inner surface 256 may also include bone growth promoting agent 258. Additionally, holes 252 may also be lined with bone growth promoting agent 258. This configuration preferably allows bone to grow along outer surface 254 as well as inner surface 256, via holes 252. Bone growth can also occur into the holes themselves, and into the bone growth promoting agent of the prosthesis.

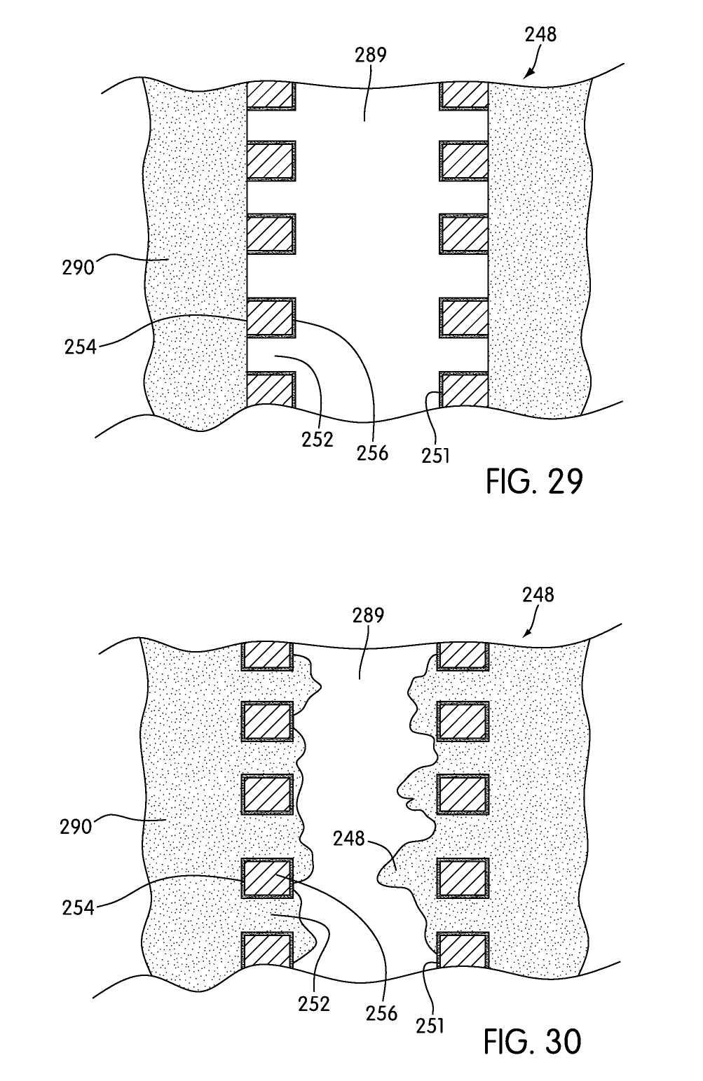

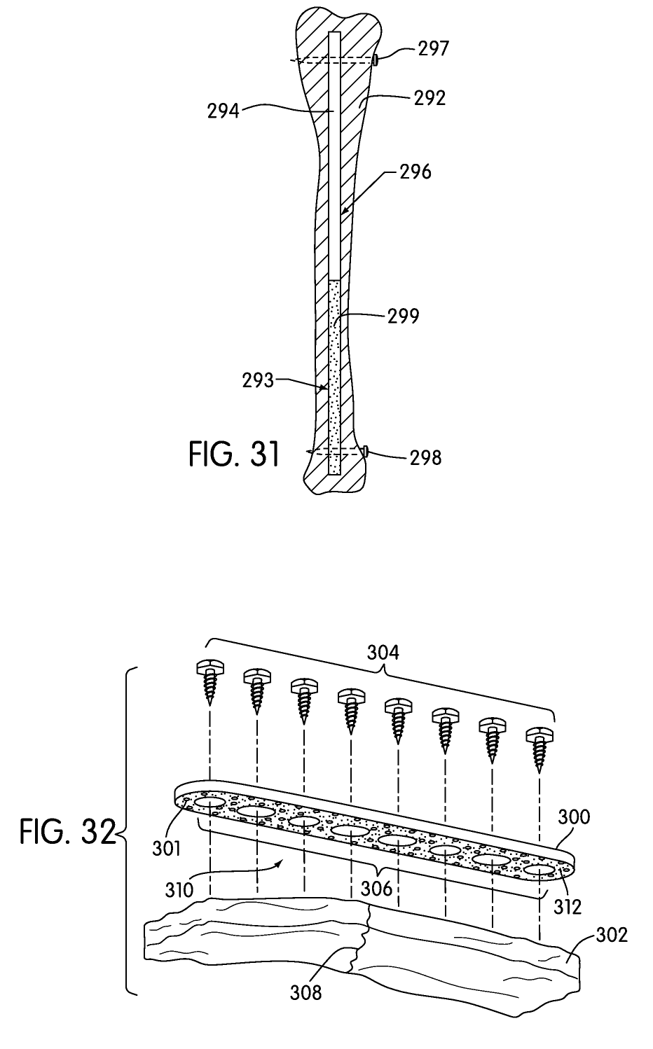

Referring to FIGS. 29-30, ingrowth of the bone from outer surface 254 to inner surface 256 may proceed once rod 248 has been inserted into a section of bone 290 or surrounded by bone 290, whether from a fracture or fusion. With time, portions 291 of bone 290 may grow through holes 252 into hollow central core 289. In some embodiments, portions 291 may fuse together inside hollow central core 289. In this way, rod 248 may be fused with bone 290. In a preferred embodiment, holes 252 are used in conjunction with bone growth promoting agent 251 disposed along inner surface 256 and outer surface 254 in order to induce bone growth. In some embodiments, bone growth promoting agent 251 may also be disposed within holes 252. In this manner, rod 248 may be partially or fully integrated into bone 290 as it heals.

Generally, in the rod embodiment disclosed above, or in any of the embodiments disclosed below, a combination of macroscopic holes and microscopic holes or other bone growth promoting surface treatments can be used. By using a combination of both features, bone growth can be encouraged at the surface of the prosthesis so that the prosthesis, on a surface level, integrates with the bone; and by using macroscopic holes, large scale or bulk integration of the prosthesis can occur, further solidifying the integration of the prosthesis with the bone.

FIG. 28 is a cross sectional view of a preferred embodiment of implantable prosthesis system 296. Preferably, implantable prosthesis system 296 is integrated into bone 292 (seen here in cross section). Preferably, implantable prosthesis system 296 may include rod 294, as well as first bone screw 297 and second bond screw 298. In some embodiments, rod 294 may include bone growth promoting agent 299, disposed along a first portion 293 of rod 294. First portion 293 can range from a relatively small portion of rod 294 to substantially all of rod 294. In some embodiments, second screw 298 may also be coated with bone growth promoting agent 299. Generally, any desired number of screws in system 296 can include bone growth promoting agents. It is also possible that the location of various, differently treated screws is varied depending on the type of bone. For example, a screw for use in cortical bone may have one type of bone growth promoting agent, while a screw for use in cancellous or spongy bone has a second type of bone growth promoting agent. In this manner, the portion of bone 292 disposed adjacent to first portion 293 of rod 294 and second screw 298 may be stimulated to grow and fuse around rod 294 and second screw 298.

In an alternative embodiment, the implantable prosthesis may take the form of a fracture plate. In a manner similar to the rods discussed in the previous embodiments, a bone growth promoting agent may be applied to a fracture plate to stimulate bone growth. In a preferred embodiment, a bone growth promoting agent may be selectively applied to various portions of a fracture plate, stimulating bone growth along various portions of the bone.

FIG. 32 is an exploded isometric view of a preferred embodiment of fracture plate 300 that may be attached to bone 302. Generally, fracture plate 300 may be attached to bone 302 using screw set 304. The screws comprising screw set 304 may be inserted through screw hole set 306 of fracture plate 300. With this arrangement, fracture plate 300 may be attached to bone 302 in order to add support to bone 302 while fracture 308 heals. Generally, any number of screws and screw holes may be used. In this exemplary embodiment, there are eight screws comprising screw set 304 and eight screw holes comprising screw hole set 306.

In the preferred embodiments, the profile of fracture plate 300 is minimized by the long and narrow shape of fracture plate 300. Additionally, the profile may be minimized by the use of large screw holes. This reduction in profile may decrease the tendency of fracture plate 300 to interfere with the surrounding tissue and may also help decrease the weight of fracture plate 300 while maintaining a high density for strength and durability.

In the preferred embodiment, fracture plate 300 may also include small holes 301 that are disposed on lower surface 310. Small holes 301 may be macro and/or micro holes. Small holes 301 may extend partially into fracture plate 300, or may extend all the way through. Also, small holes 301 may be disposed anywhere on lower surface 310, in any pattern, including a random pattern. The use of small holes 301 preferably facilitates both macro and micro fixation of bone growth.

In some embodiments, fracture plate 300 may include a lower surface 310. In some embodiments, lower surface 310 may be coated with bone growth promoting agent 312. Preferably, in this embodiment, bone growth promoting agent 312 may cover the entirety of lower surface 310. Generally, bone growth promoting agent 312 may be any of the types of bone growth promoting agents discussed previously.

In some embodiments, an intermediate tissue or membrane is disposed between fracture plate 300 and bone 302. In other words, fracture plate 300 may not directly contact bone 302. Instead, fracture plate 300 may be configured to contact some other tissue or membrane disposed adjacent to bone 302. This membrane can include muscle or periosteum.

As with the rods in the previous embodiments, bone growth promoting agents may be selectively applied to various portions of fracture plates. In this way, different portions of a bone in contact with a fracture plate may be stimulated to grow differently. Generally, a bone growth promoting agent may be applied to any portion of a fracture plate. Additionally, a bone growth promoting agent may be disposed in any pattern along the fracture plate. This may be useful in cases where some, but not all, portions of the bone are damaged.

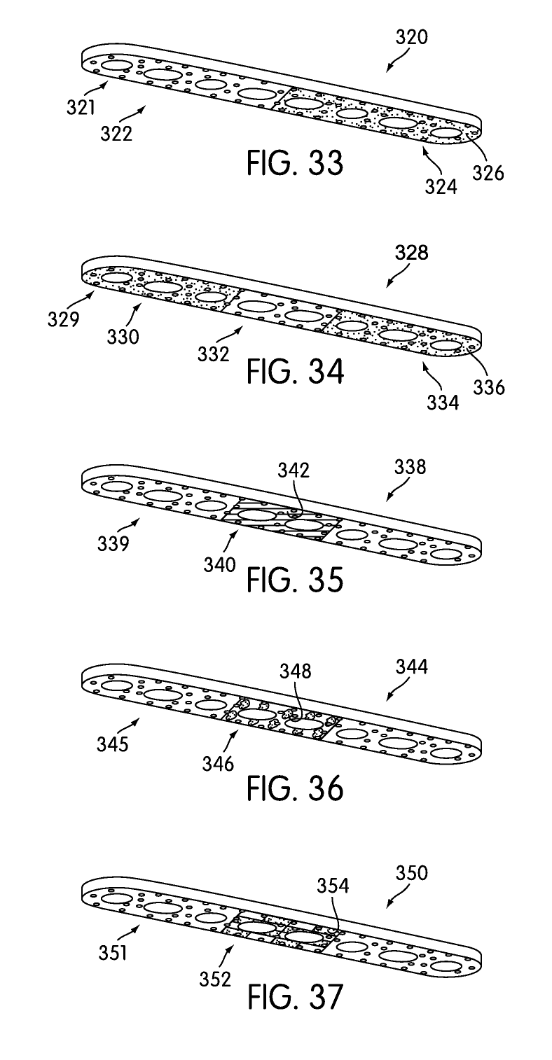

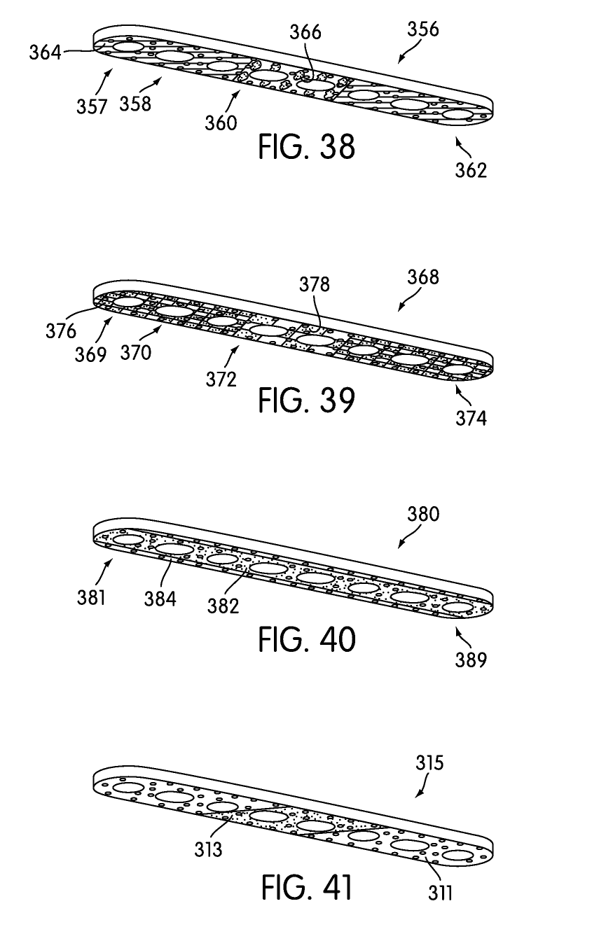

Referring to FIGS. 32-41, bone growth promoting agents may be applied to a fracture plate in a variety of ways. The following embodiments are intended to illustrate possible configurations of fracture plates including one or more bone growth promoting agents, however it should be understood that these embodiments are only intended to be exemplary. Many other types of bone growth promoting agents, including various patterns may be applied to one or multiple portions of a fracture plate. Additionally, throughout the following embodiments, the bone growth promoting agents may be used in combination with macro and micro holes in order to further facilitate bony fusion.

First plate 320 preferably includes first lower surface 321. In some embodiments, first lower surface 321 may include first portion 322 and second portion 324. In some embodiments, first portion 322 and second portion 324 may have different treatments. In a preferred embodiment, first portion 322 is not treated. In a preferred embodiment, second portion 324 may be treated with bone growth promoting agent 326.

As previously discussed, bone growth promoting agent 326 may include chemical treatments of the surface, or modifications to the texture of the surface of the prosthesis. Generally, the bone growth promoting agent applied to a fracture plate may be any type of bone growth promoting agent discussed in the previous embodiments involving rods, as well as any other bone growth promoting agent. In these embodiments, the bone growth promoting agents are visually distinct from the general surface to which they are applied. However, this is done purely for illustrative purposes. In some embodiments, the bone growth promoting agents may not be visible.

Second fracture plate 328 also preferably includes several portions. In some embodiments, second plate 328 may include lower surface 329. In some embodiments, second lower surface 329 may include first portion 330, second portion 332, and third portion 334. In some embodiments, first portion 330 and third portion 334 may be treated in a similar manner. In a preferred embodiment, first portion 330 and third portion 334 both include bone growth promoting agent 336. In this manner, second fracture plate 328 preferably helps to induce growth along portions of the bone adjacent to first portion 330 and third portion 334, but not second portion 332.

Additionally, fracture plates may be treated with a bone growth promoting agent that is disposed along the outer surface in a variety of designs. These designs may be similar to the designs discussed in previous embodiments, or other types of designs. In some embodiments, fracture plates may include a bone growth promoting agent applied in striped, spotted, geometric patterns, and/or combinations of two or more of these basic patterns.

Third fracture plate 338 preferably includes center portion 340 disposed along lower surface 339. In some embodiments, center portion 340 may include a bone growth promoting agent. In a preferred embodiment, center portion 340 includes bone growth promoting agent 342 configured in a striped pattern.

In another embodiment, fourth fracture plate 344 also preferably includes center portion 346 disposed along lower surface 345. In some embodiments, center portion 346 may include a bone growth promoting agent. In a preferred embodiment, center portion 346 may include bone growth promoting agent 348 configured in a spotted pattern.

In another embodiment, fifth fracture plate 350 also preferably includes center portion 352 disposed along lower surface 351. In some embodiments, center portion 352 may include a bone growth promoting agent. In a preferred embodiment, center portion 352 preferably includes bone growth promoting agent 354 configured in a geometric pattern.

In another embodiment, sixth fracture plate 356 may include three separate portions. Preferably, sixth fracture plate 356 includes first portion 358, second portion 360, and third portion 362 disposed along lower surface 357. In some embodiments, each portion may be treated with a different bone growth promoting agent. In some embodiments, first portion 358 and third portion 362 may be treated with a similar pattern of bone growth promoting agent. In a preferred embodiment, first portion 358 and third portion 362 may include bone growth promoting agent 364 configured in a striped pattern. Also, second portion 360 may preferably include bone growth promoting agent 366 configured in a spotted pattern.

In some cases, different portions may be treated with the same pattern of bone growth promoting agents, but the size or density of the pattern may differ between portions. Seventh fracture plate 368 preferably includes several portions disposed along lower surface 369. In particular, seventh fracture plate 368 preferably includes first portion 370, second portion 372, and third portion 374. In some embodiments, each of these portions 370, 372 and 374 may include a bone growth promoting agent disposed in a geometric pattern. In a preferred embodiment, first portion 370 and third portion 374 may include a first bone growth promoting agent 376 disposed in a high density geometric pattern. Likewise, second portion 372 may include a second bone growth promoting agent 378 disposed in a low density geometric pattern.

In the previous embodiments, a bone growth promoting agent was applied along portions that were disposed along the width of the fracture plates. In some embodiments, however, the bone growth promoting agent may be disposed along portions that are oriented along the length of the fracture plates. Additionally, a fracture plate may be divided into several portions disposed along the length of the fracture plate, each portion including a different type of bone growth promoting agent.

FIG. 40 is a preferred embodiment of fracture plate 380. In some embodiments, fracture plate 380 may include lower surface 381. In some embodiments, lower surface 381 may be coated with bone growth promoting agent 382 along vertical portion 389. FIG. 41 illustrates an embodiment of a fracture plate. In this embodiment, fracture plate 315 includes a diagonally applied bone growth promoting agent 313 onto lower surface 311. Using either a vertically or diagonally applied bone growth promoting agent may facilitate new bone growth along the length of a fracture plate.

In some embodiments, a fracture plate may include additional provisions for inducing bone growth, such as a porous surface. Additionally, fracture plate 380 may include holes 384 disposed along lower surface 381. Generally, holes 384 may have circumferences of various sizes. Likewise, holes 384 may have various depths. Holes 384 need not be disposed along the entirety of fracture plate 380. In some embodiments, holes 384 may be confined to one or multiple portions of a fracture plate. As disclosed above, fracture plate 380 is an example of a prosthesis that includes both macroscopic holes 384 and microscopic bone growth promoting features or agents 382. These macroscopic and microscopic features can be used in combination to help integrate fracture plate 380 to the bone in a macroscopic and microscopic scale.

In another embodiment, a fracture plate may include a liner. In some embodiments, the liner may fit into a recess disposed in the fracture plate. However, in other embodiments, no recess is provided for the liner. Generally, the liner may be formed of or coated with a bone growth promoting agent. The bone growth promoting agent may be disposed on the liner in any pattern, such as those patterns described above with respect to the fracture plate. In this manner, a liner with a bone growth promoting agent may be manufactured separately from the fracture plate, and combined with the fracture plate at the time of surgery, during implantation, or after implantation. It is also possible to provide a fracture plate with a pre-installed liner so there is no need for the surgeon to associate the liner with the fracture plate at the time of surgery.

In some embodiments, the liner may be attached to the fracture plate through an adhesive. It is also possible to attach the liner to the fracture plate by using mechanical provisions, including hooks, microscopic hooks, temperature difference, interference fit or a Morris taper. It is also possible to attach the liner to the fracture plate using magnetic features. In some embodiments, liner may be preconfigured to include an adhesive for attaching to the fracture plate.

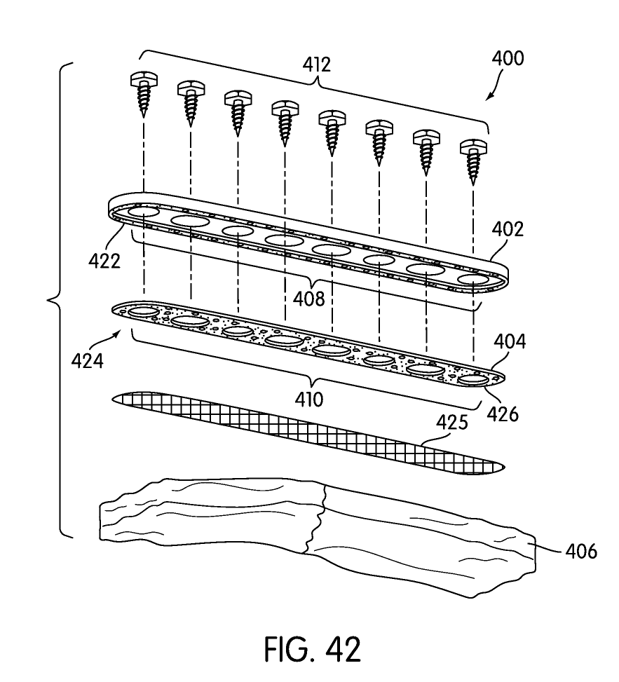

FIG. 42 is an exploded view of a preferred embodiment of liner system 400. Liner system 400 preferably includes fracture plate 402. Preferably, fracture plate 402 includes lower surface 422. In some embodiments, recess 420 may be disposed along lower surface 422 of fracture plate 402. Recess 420 may include second set of holes 408.

Additionally, liner system 400 also preferably includes liner 404. Liner 404 may be made of a similar material to fracture plate 402. In some embodiments, liner 404 may be a wafer of bone. Using a wafer of bone may help facilitate bone to bone fusion. In some embodiments, liner 404 may include lower surface 424. Preferably, lower surface 424 includes bone growth promoting agent 426. In a preferred embodiment, lower surface 424 is disposed adjacent to bone 406. Liner 404 also preferably includes first set of holes 410.

In some embodiments, liner system 400 may also include mesh 425. Generally, mesh 425 may be treated with a bone growth promoting agent. In some embodiments, mesh 425 may be disposed between liner 404 and bone 406. In other embodiments, liner system 400 may include only mesh 425 or liner 404. In some embodiments, mesh 425 may be a bone wafer, composite, bio-compatible material or a second liner.

In some embodiments, fracture plate 402 may be constructed of a bio-absorbable material. In this manner, fracture plate 402 may eventually dissolve into the tissue surrounding it. This is a preferred situation over situations in which the fracture plate would need to be removed via surgery. In a similar manner, the fracture plate 402, the liner 404 and/or the mesh 425 may be constructed of a bio-absorbable material. Liner 404 and/or mesh 425 can be constructed of bone, collagen or other biological or bio-compatible materials. In some cases, a bone wafer may be used. Additional liners and/or meshes may be used, resulting in more than two liners and possibly more than two meshes.

Generally, recess 420 may be configured to receive liner 404. In some embodiments, recess 420 has a depth that is equivalent to the thickness of liner 404. In other embodiments, the thickness of liner 404 and the depth of recess 420 may be varied.

Preferably, liner system 400 also includes screw set 412. In some embodiments, second set of holes 408 are configured to receive screw set 412. Generally, first set of holes 410 and second set of holes 408 may be aligned.

Once assembled, liner system 400 may be configured to add support to bone 406. In particular, as liner 404 preferably includes selectively applied bone growth promoting agent 426 along lower surface 424, this may help stimulate the growth of bone 406. Generally, a liner may also include various bone growth promoting agents that may be selectively applied to various regions. The types of bone growth promoting agents and the methods of selectively applying them may be substantially similar to the previous embodiments.

In some embodiments, a fracture plate with holes may help induce bone growth that allows bone to grow into the holes. In this manner, the bone may be partially fused to the fracture plate. Preferably, the plate may include an additional bone growth promoting agent to help stimulate bone growth.

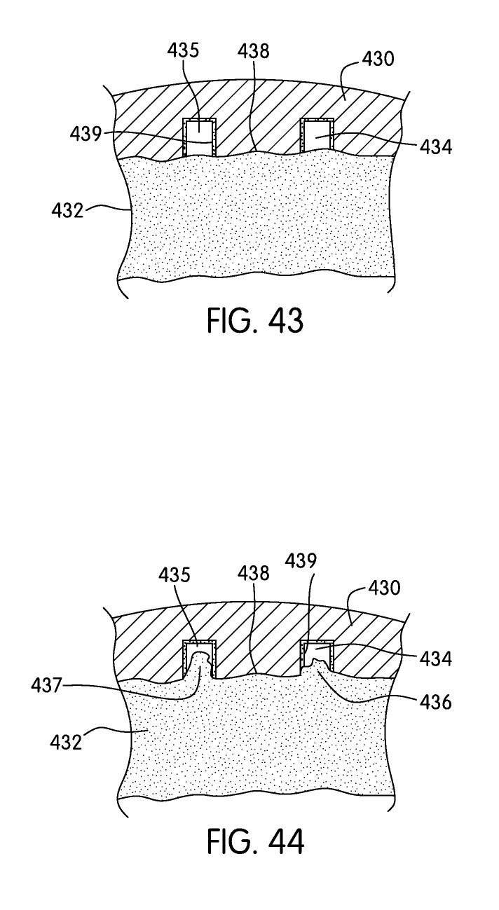

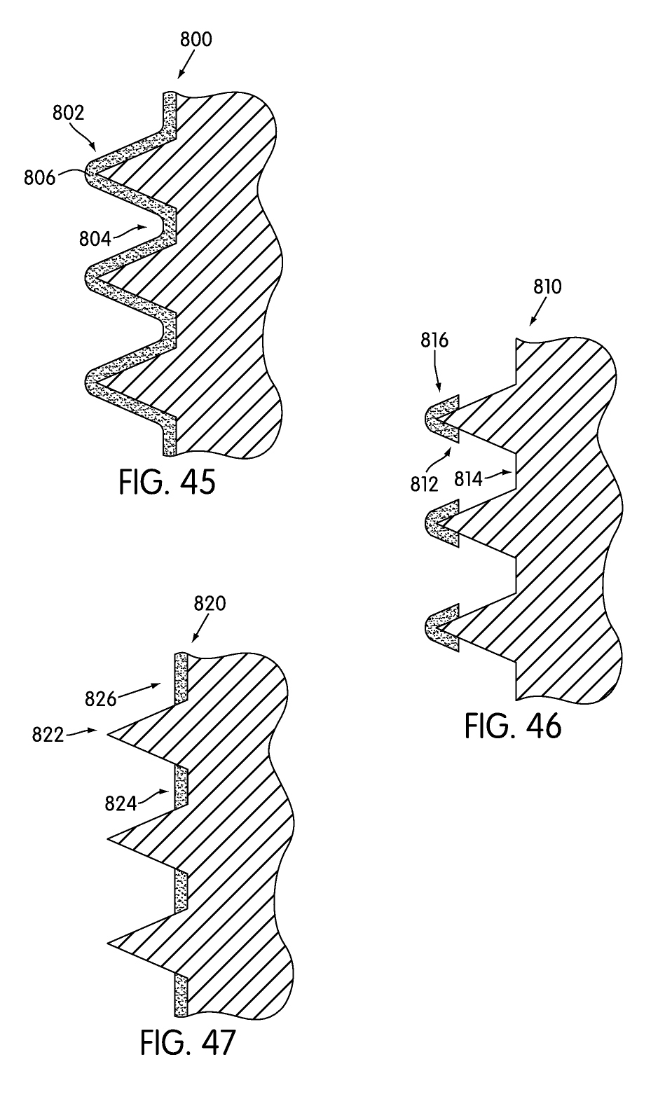

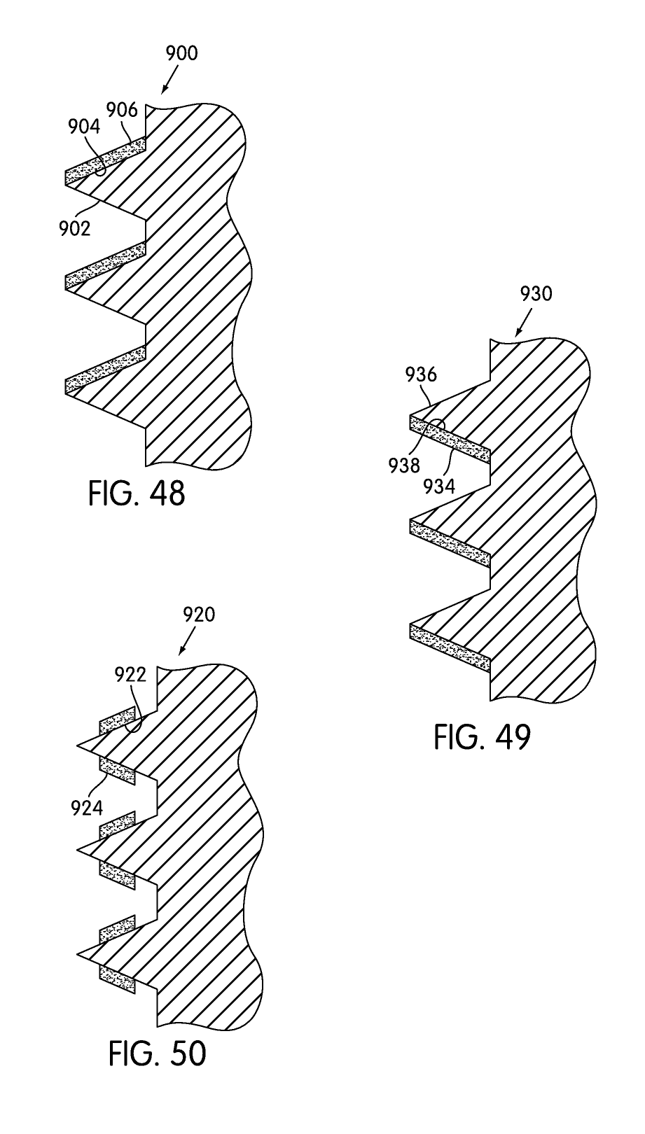

Referring to FIGS. 43-44, fracture plate 430 may preferably be configured to promote bone growth on the walls of first hole 434, second hole 435, and lower surface 438. This may be achieved with or without the use of a bone growth promoting agent. In a preferred embodiment, bone growth promoting agent 439 may be applied to holes 434 and 435. Generally, fracture plate 430 may be secured to bone 432 by some means, such as a screw. Over time, first portion 436 and second portion 437 of bone 432 may grow into first hole 434 and second hole 435. In addition, bone growth will also occur into the surfaces of first hole 434 and second hole 435. In other words, bone growth can occur on a macroscopic scale—bone growth into holes 434 and 435—and on a microscopic scale as well, bone growth onto the surfaces of holes 434 and 435 due to the bone growth promoting agent applied to the walls of holes 434 and 435.

In an alternative embodiment, the implantable prosthesis may take the form of a screw. In some cases, a screw may be configured to attach multiple bones together. In other cases, a screw may be configured to attach a rod or a fracture plate to a fractured single bone. Generally, a screw may be used with many different kinds of implantable prostheses.

In a manner similar to the rods and fracture plates discussed in the previous embodiments, a bone growth promoting agent may be selectively applied to a screw to stimulate bone growth. Because a screw has a similar structure to a rod, it follows that all of the various modifications that may be made to a rod to include selectively applied bone growth promoting agents may also be applied to the screw disclosed here. In particular, any of the bone growth agents previously disclosed may be applied to any portion of a screw. Also, these bone growth agents may be applied in the patterns disclosed in the previous embodiments.

The term screw as used here applied to any device with threading. In some cases, screws may or may not include a head. Screws can also include a solid or hollow boring tip. This solid boring tip allows the screw to be inserted into a region of bone where no previous hole has been made. Additionally, the head may be associated with a fastening tool, such as a screw driver, hex key or a drill, allowing the screw to be turned.

In FIG. 45, bone growth promoting agent 806 has been applied to threading peaks 802 of threading 800 as well as threading valleys 804 of threading 800. This coating of the entirety of threading 800 may be accomplished by dipping threading 800 in a chemical including bone growth promoting agent 806. The coating can also be applied by spraying, sintering, wax covering, as well as other suitable methods.

Additionally, it may be desirable in some cases to only coat a portion of the threading. This can provide different degrees of incorporation into the bone. In some cases, limited degrees of incorporation may be helpful to assist in later removal of the screw. Referring to FIG. 46, it may be possible to only apply bone growth promoting agent 816 to threading peaks 812 of threading 810. In this manner, threading valleys 814 may not include bone growth promoting agent 816. This feature may be accomplished by quickly dipping threading 810 into a chemical including bone growth promoting agent 816 before the chemical has time to fill into thread valleys 814. Additionally, the coating can also be applied by spraying, sintering, wax covering, as well as other suitable methods.

In some cases, only the threading valleys may be coated. Referring to FIG. 47, threading valleys 824 of threading 820 may be coated with bone growth promoting agent 826. This may be accomplished by dipping threading 820 into a chemical including bone growth promoting agent 826, and then spinning the screw in a manner that expels the bone growth promoting agent 826 from threading peaks 822. Additionally, the coating can also be applied by spraying, sintering, wax covering, as well as other suitable methods.

In other embodiments, only portions of the threading may be coated. Referring to FIG. 48, threading 900 preferably includes upper portions 904 and lower portions 902. In this embodiment, only upper portions 904 of threading 900 may be coated with bone growth promoting agent 906. Likewise, in the embodiment shown in FIG. 49, threading 930 may include upper portions 936 and lower portions 938. In this embodiment, only lower portions 938 of threading 930 may be coated with bone growth promoting agent 934. Finally, in the embodiment shown in FIG. 50, only middle portions 922 of threading 920 may be coated with bone growth promoting agent 924. As with the previous embodiments, each of the coatings may be applied using techniques such as spraying, sintering, wax covering, as well as other suitable techniques.

In some embodiments, the structure of a screw may be modified. Such modifications include hollowing out the screw, as well as adding holes to the screw. Generally, a screw may be modified in ways similar to the rods disclosed above. The screws may be fully, partially or non-cannulated screws and the coatings may be applied in whole or in part in a manner similar to the coatings applied to the rods as disclosed above.

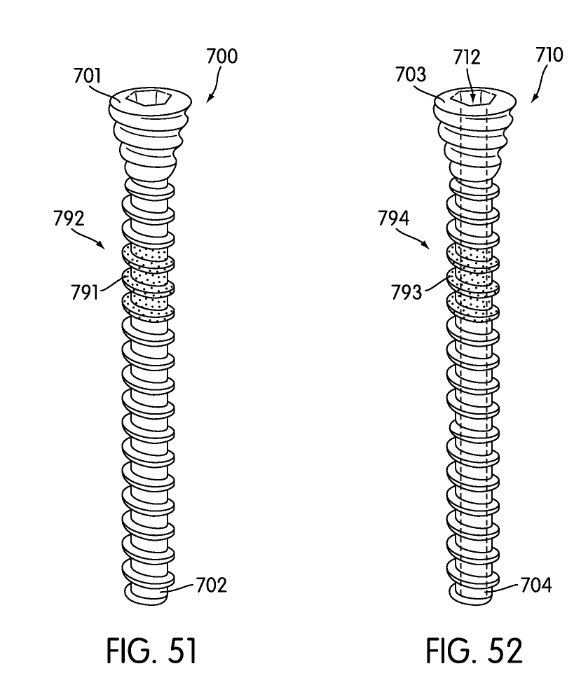

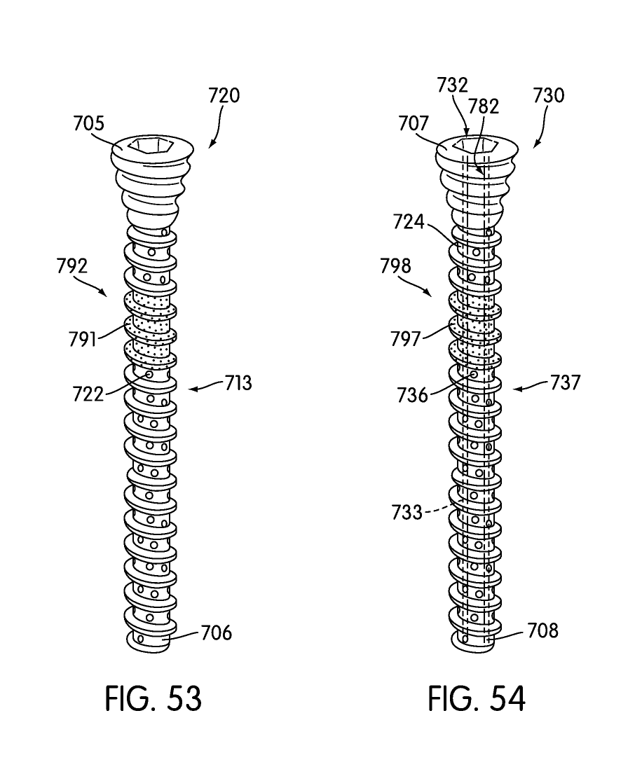

Referring to FIGS. 51-54, screws may be configured solid, hollow, and with or without holes. One example of a hollow screw is a cannulated screw, which includes a hollow central shaft. In one embodiment, a section of screw 700 may be solid. Screw 700 also preferably includes screw head 701 and boring tip 702. In some embodiments, bone growth promoting agent 791 may be applied to first region 792. Preferably, bone growth promoting agent 791 is only applied to first region 792 and not the entire shaft of screw 700. Likewise, throughout the remaining embodiments seen in FIGS. 52-54, bone growth promoting agents have been applied only to a selected region of the screw, not to the entirety. In this manner, screw 700 may stimulate bone growth along portions of a bone disposed adjacent to first region 792.

In a second embodiment, screw 710 may include hollow central core 712. Second screw 710 may include screw head 703 and boring tip 704. In some embodiments, bone growth promoting agent 793 may be applied to first region 794. In this manner, screw 710 may stimulate bone growth along portions of a bone disposed adjacent to first region 794.

Preferably, in a third embodiment, screw 720 may include holes 722. Holes 722 are preferably disposed along a first portion 713 of screw 720. Generally, holes 722 may be any depth, any shape, angle, and have any size circumference. Similarly, the density of holes 722 may be varied in other embodiments. In some embodiments, a combination of holes having different sizes, shapes, angles or densities may be used. Preferably, screw 720 may also include screw head 705 and boring tip 706. In some embodiments, bone growth promoting agent 795 may be applied to second portion 796. In this manner, screw 720 may stimulate bone growth along portions of a bone disposed adjacent to first region 796. In a preferred embodiment, a bone growth promoting agent is not applied to screw head 705.

A fourth embodiment of a section of screw 730 may preferably include hollow central core 732 as well as holes 736. Holes 736 are preferably disposed along first portion 737 of screw 730. Generally, holes 736 may be any depth, any shape, angle, and have any size circumference. Similarly, the density of holes 736 may be varied in other embodiments. In some embodiments, a combination of holes having different sizes, shapes, angles or densities may be used. In a preferred embodiment, holes 736 may be disposed between outer surface 729 and inner surface 733 of hollow central core 732. In this manner, holes 736 preferably allow fluid communication between hollow central core 732 and outer surface 729. Preferably, fourth screw 730 may also include screw head 707 and boring tip 708. In some embodiments, bone growth promoting agent 797 may be applied to first region 798. In this manner, screw 730 may stimulate bone growth along portions of a bone disposed adjacent to first region 798. In a preferred embodiment, inner surface 733 may include bone growth promoting agent 782 as well. Bone growth promoting agent 782 applied to inner surface 733 may be similar or different than bone growth promoting agent 797 that is applied to first region 798. The various bone growth promoting agents can be selected to achieve different bone growth properties and/or to encourage different rates or kinds of bone growth. In a preferred embodiment, a bone growth promoting agent is not applied to screw head 707.

Generally, the length of the central cavities 712 and 732 of the previous embodiments may be varied. Preferably, central cavities 712 and 732 extend all the way to the bottom of screws 710 and 730. Instead, the end of screws 710 and 730 are preferably solid, as is preferable for boring into bone. Additionally, the tops of screws 710 and 730 need not be configured open. In some embodiments, the tops of screws 710 and 730 may be configured closed. Furthermore, screw heads in any embodiment may include features to mate with any desired driver. For example, the screw heads may include a slot, Phillips, star, hexagonal cavity, torx, hexagonal nut or any other desired mechanical coupling. In other embodiments, the screw does not have a head, and the shaft includes features to mate with any desired driver.

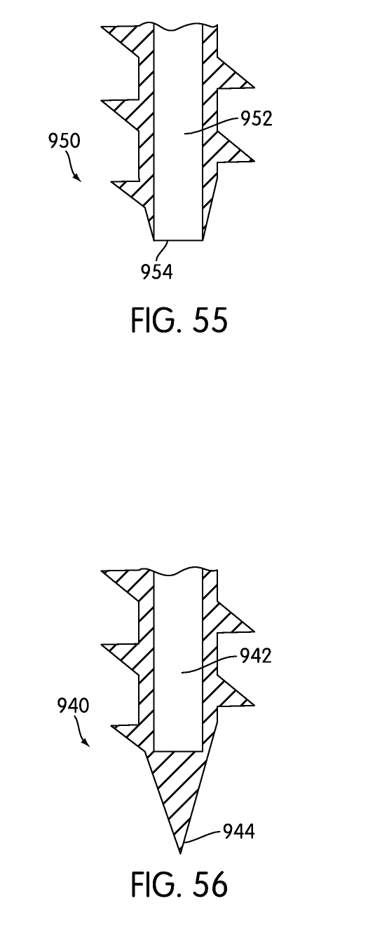

Additionally, in some embodiments, the tips of the screws including bone growth promoting agents may be configured as open or closed. In other words, the tips may have a hollow or solid boring tip. Referring to FIG. 55, a screw including tip portion 950 includes central cavity 952 that extends all the way through boring tip 954. In another embodiment, seen in FIG. 56, a screw including tip portion 940 includes central cavity 942 with a solid boring tip 944.

In a manner similar to the rods and cages of the previous embodiments, a screw may be configured to promote ingrowth of bone and fuse with the bone. In some embodiments, a screw including holes and a hollow central core may be implanted into a bone. Once the screw has been implanted inside the bone, growth may occur through the holes into the hollow central core. In a preferred embodiment, the outer and inner surfaces of the screw may be coated with a bone growth promoting agent.

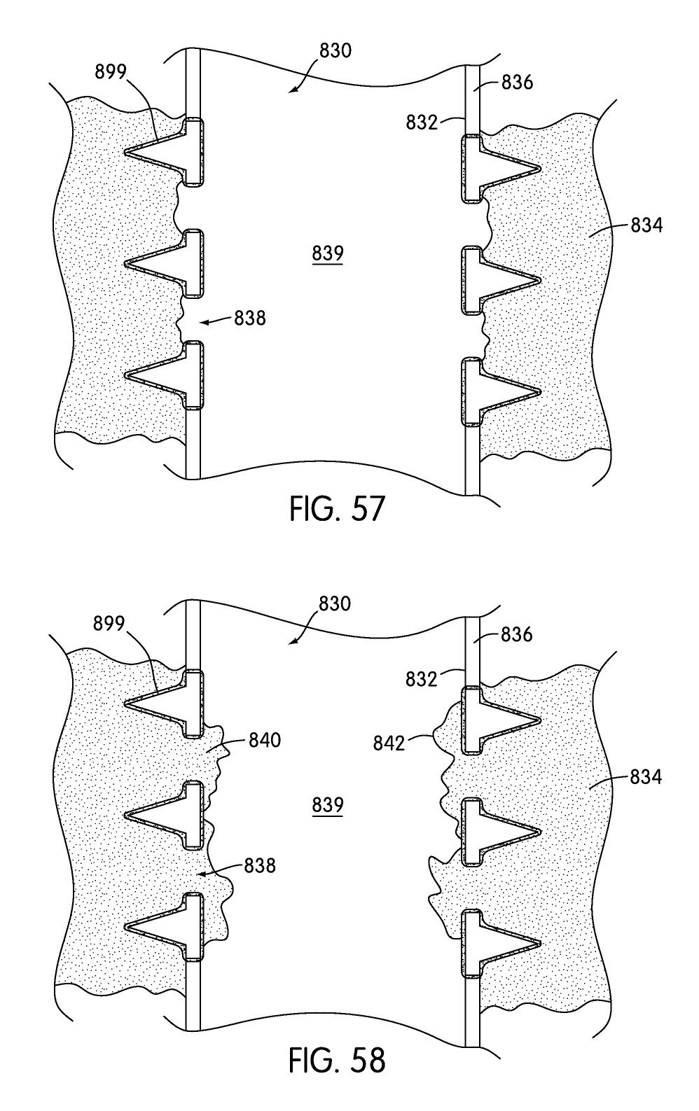

Referring to FIGS. 57-58, ingrowth of the bone from outer surface 836 to inner surface 832 may proceed once screw 830 has been inserted into a section of bone 834. With time, portions 840 of bone 834 may grow through holes 838 into hollow central core 839. In some embodiments, portions 840 may fuse together within hollow central core 839. In this way, screw 830 may be fused with bone 834. In a preferred embodiment, holes 838 are used in conjunction with bone growth promoting agent 899 disposed along inner surface 832 and outer surface 836 in order to induce bone growth. In some embodiments, bone growth promoting agent 899 may also be disposed within holes 838. In this manner, screw 830 may be partially or fully integrated into bone 834 as it is healing, micro and macroscopically.

Typically, a spinal fusion device may be inserted between adjacent vertebrae in cases where an intervertebral disc has ruptured or degenerated. In some embodiments, a portion of the intervertebral disc may be removed prior to the insertion of the spinal fusion device. Generally, spinal fusion devices configured for insertion between vertebrae include spinal implants, spinal wedges, spinal plugs and other implantable devices. The spinal fusion devices discussed throughout this detailed description may be used with any type of vertebrae, including cervical, thoracic, and lumbar vertebrae.

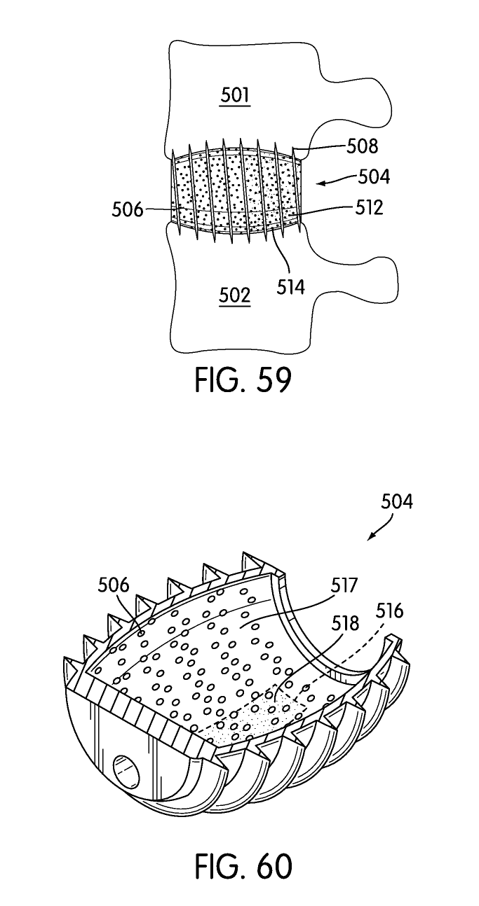

FIG. 59 is a preferred embodiment of spinal implant 504. For clarity, the following detailed description discusses a preferred embodiment, however, it should be kept in mind that the present invention could also take the form of any other kind of spinal fusion device including, for example, wedges, plugs, discs, as well as other kinds of spinal implants.

In a preferred embodiment, spinal implant 504 may be fish shaped or barrel shaped. Using a fish shaped or barrel shaped screw may help to create lordosis (an increased curvature in the lower spine). Generally, spinal implant 504 may be implanted between first vertebra 501 and second vertebra 502. Preferably, a cylindrical hole may be drilled or reamed into the intervertebral disc prior to the insertion of spinal implant 504, ensuring that portions of the first and second vertebrae 501 and 502 are also removed. In some embodiments, an appropriate diameter reamer may be used so that part of the bone on either side of the disc is removed as well. In a preferred embodiment, spinal implant 504 may be inserted between first vertebra 501 and second vertebra 502 in a manner so that threading 508 engages vertebrae 501 and 502.

Generally, a spinal fusion device, including a spinal implant, may be hollow. This hollow configuration may reduce the overall weight and density of the spinal fusion device, as opposed to a solid spinal fusion device. In other embodiments, the spinal fusion implant may include an internal lattice or spoke-like structure for increased support without a significant increase in overall weight. In still other embodiments, the spinal fusion device could have a solid core. In other words, the spinal fusion device may not be hollow in some embodiments.

Spinal fusion devices, such as spinal implants, may include provisions for increasing bony fusion. In some embodiments, a spinal fusion device may include holes. In some embodiments, the number, size, shape and density of the holes may vary. In some cases, a combination of macroscopic holes and microscopic holes or other bone growth promoting surface treatments can be used. By using a combination of both features, bone growth can be encouraged at the surface of the spinal fusion device so that the spinal fusion device, on a surface level, integrates with the bone; and by using macroscopic holes, large scale or bulk integration of the spinal fusion implant can occur, further solidifying the integration of the spinal fusion implant with the bone. Furthermore, in some embodiments, some or all of the holes may penetrate through the surface of the spinal fusion device into a hollow central core. In other embodiments, the holes may or may not penetrate through the surface of the spinal fusion implant. In other words, the holes may have bottoms.

Spinal implant 504 may include holes 506. In this embodiment, holes 506 have a spacing that is large compared to their diameter. In other embodiments, holes 506 may be spaced closer together, in a honeycomb configuration for example. Holes 506 may be configured so that portions of adjacent bone may grow through spinal implant 504. Using this configuration, spinal implant 504 may provide support and may facilitate the fusion of first vertebra 501 to second vertebra 502. It should be understood that although the preferred embodiment discussed here includes holes, in other embodiments, spinal implant 504 may not include any holes.

Generally, a bone growth promoting agent may be selectively applied to a portion of a spinal fusion device, such as a spinal implant. In some embodiments, a bone growth promoting agent may be selectively applied along a portion of the outer surface of the spinal fusion device. In embodiments that include an inner surface, a bone growth promoting agent may be selectively applied along a portion of the inner surface. Additionally, in some embodiments, a bone growth promoting agent may be applied to macroscopic and/or microscopic holes which may or may not penetrate through the surface of the spinal fusion device. Using this configuration, a bone growth promoting agent may be applied to different portions of the spinal fusion device in order to help promote bone growth differently along different portions of the adjacent bone.

In some embodiments, spinal implant 504 may include first portion 512, as seen in FIG. 59. In a preferred embodiment, first portion 512 may include bone growth promoting agent 514. With this configuration, bone adjacent to first portion 512 may be induced to grow through holes 506 along first portion 512 adjacent to the bone, which may facilitate in fusing spinal implant 504 with vertebrae 501 and 502. This may be useful in situations where the surgeon only wants to stimulate bone growth at particular portions of vertebrae 501 and 502.

In this embodiment, bone growth promoting agent 514 has been selectively applied to first portion 512 of spinal implant 504. However, in other embodiments, bone growth promoting agent 514 may be selectively applied to any portion of spinal implant 504. In some embodiments, bone growth promoting agent 514 may be selectively applied to all portions of spinal implant 504.

Referring to FIG. 60, spinal implant 504 preferably includes inner surface 517. For illustrative purposes, FIG. 60 only includes the lower half of spinal implant 504; however spinal implant 504 also comprises a second half not shown here. Preferably, holes 506 are also disposed along inner surface 517. In other words, holes 506 generally penetrate through spinal implant 504.

In some embodiments, inner surface 517 may include first portion 516. In a preferred embodiment, first portion 516 of inner surface 517 may include bone growth promoting agent 518. With this configuration, bone may be induced to grow through holes 506 within first portion 516.

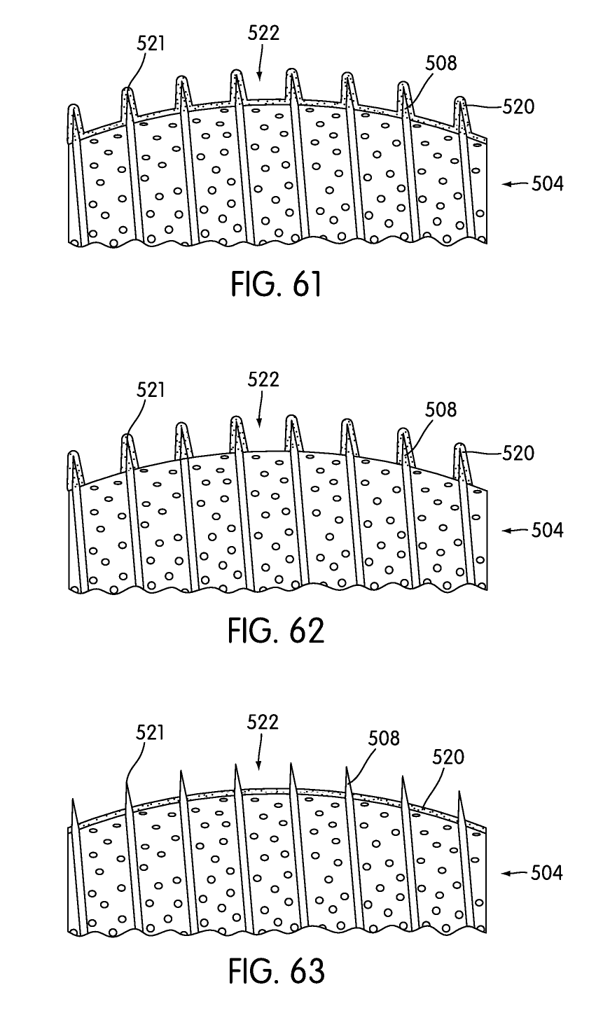

In some embodiments, a bone growth promoting agent may also be selectively applied to portions of threading 508. As threading 508 preferably engages the adjacent vertebrae, a bone growth promoting agent along portions of threading 508 may facilitate bone growth in the adjacent vertebrae. In FIG. 61, bone growth promoting agent 520 has been applied to threading peaks 521 of threading 508 as well as threading valleys 522 of threading 508. As an example, this coating of the entirety of threading 508 may be accomplished by dipping threading 508 in a chemical including bone growth promoting agent 520. In other embodiments, the coating of threading 508 may be accomplished using a plasma spray or a similar kind of chemical treatment.

Additionally, it may be desirable in some cases to only coat a portion of threading 508. Referring to FIG. 62, it may be possible to only apply bone growth promoting agent 520 to threading peaks 521 of threading 508. With this arrangement, threading valleys 522 may not include bone growth promoting agent 520. As an example, this feature may be accomplished by quickly dipping threading 508 into a chemical including bone growth promoting agent 520. By dipping threading 508 quickly, no time is allowed for the chemical to fill threading valleys 522. In other embodiments, the coating of threading 508 may be accomplished using a plasma spray or a similar kind of chemical treatment.

In some cases, only threading valleys 522 may be coated. Referring to FIG. 63, threading valleys 522 of threading 508 may be coated with bone growth promoting agent 520. As an example, this may be accomplished by dipping threading 508 into a chemical including bone growth promoting agent 520, and then spinning spinal implant 504 in a manner that expels bone growth promoting agent 520 from threading peaks 521. In other embodiments, the coating of threading 508 with bone growth promoting agent 520 may be achieved using a plasma spray or using other kinds of chemical treatment techniques.

FIGS. 59-63 are only meant to be illustrative of the various ways a bone growth promoting agent could be selectively applied to a spinal implant. In other embodiments, various other portions of the spinal implant, including portions of an inner surface and the threading, may include bone growth promoting agents. Generally, bone growth promoting agents may be selectively applied throughout any portion of the spinal implant using any pattern, including the various types of patterns discussed at the beginning of this detailed description for rods and plates.

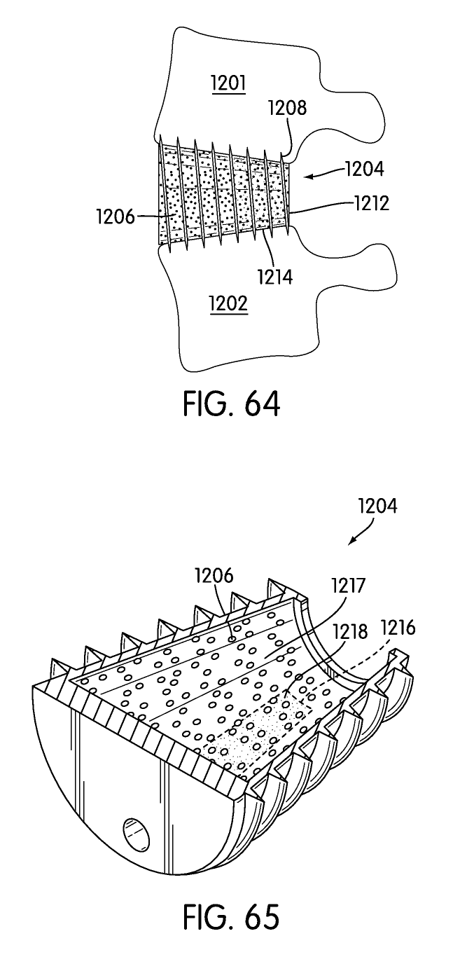

In another embodiment, shown in FIG. 64, spinal implant 1204 may be conical in shape. Using a conically shaped screw may help in creating lordosis. Generally, spinal implant 1204 may be implanted between first vertebra 1201 and second vertebra 1202. Preferably, a cylindrical hole may be drilled or reamed into the intervertebral disc prior to the insertion of spinal implant 1204, ensuring that portions of the first and second vertebrae 1201 and 1202 are also removed. In some embodiments, an appropriate diameter reamer may be used so that part of the bone on either side of the disc is removed as well. In a preferred embodiment, spinal implant 1204 may be inserted between first vertebra 1201 and second vertebra 1202 in a manner so that threading 1208 engages these vertebrae 1201 and 1202.

Preferably, spinal implant 1204 may include holes 1206. In some embodiments, the number, size and spacing of holes 1206 may vary. In this embodiment, holes 1206 have a spacing that is large compared to their diameter. In other embodiments, holes 1206 may be spaced closer together, in a honeycomb configuration, for example. Holes 1206 may be configured so that portions of adjacent bone may grow through spinal implant 1204. Using this configuration, spinal implant 1204 may provide support and may facilitate the fusion of first vertebra 1201 to second vertebra 1202. Although the preferred embodiment discussed here includes holes, in other embodiments, spinal implant 1204 may not include any holes.

In some embodiments, spinal implant 1204 may include first portion 1212, as seen in FIG. 64, which is disposed over a front portion of spinal implant 1204. In a preferred embodiment, first portion 1212 may include bone growth promoting agent 1214. With this configuration, bone adjacent to first portion 1212 may be induced to grow through holes 1206 along first portion 1212, which may facilitate fusing spinal implant 1204 with vertebrae 1201 and 1202. This may be useful in situations where the surgeon only wants to stimulate bone growth at particular portions of vertebrae 1201 and 1202.

In this embodiment, bone growth promoting agent 1214 has been selectively applied to first portion 1212 of spinal implant 1204. However, in other embodiments, bone growth promoting agent 1214 may be selectively applied to any portion of spinal implant 1204. In some embodiments, bone growth promoting agent 1214 may be selectively applied to all portions of spinal implant 1204.

Referring to FIG. 65, spinal implant 1204 preferably includes inner surface 1217. For illustrative purposes, FIG. 65 only includes the lower half of spinal implant 1204, however spinal implant 1204 also comprises a second half not shown here. Preferably, holes 1206 are also disposed along inner surface 1217. In other words, holes 1206 generally penetrate through spinal implant 1204.

In some embodiments, inner surface 1217 may include first portion 1216. In a preferred embodiment, first portion 1216 of inner surface 1217 may include bone growth promoting agent 1218. With this configuration, bone may be induced to grow through holes 1206.

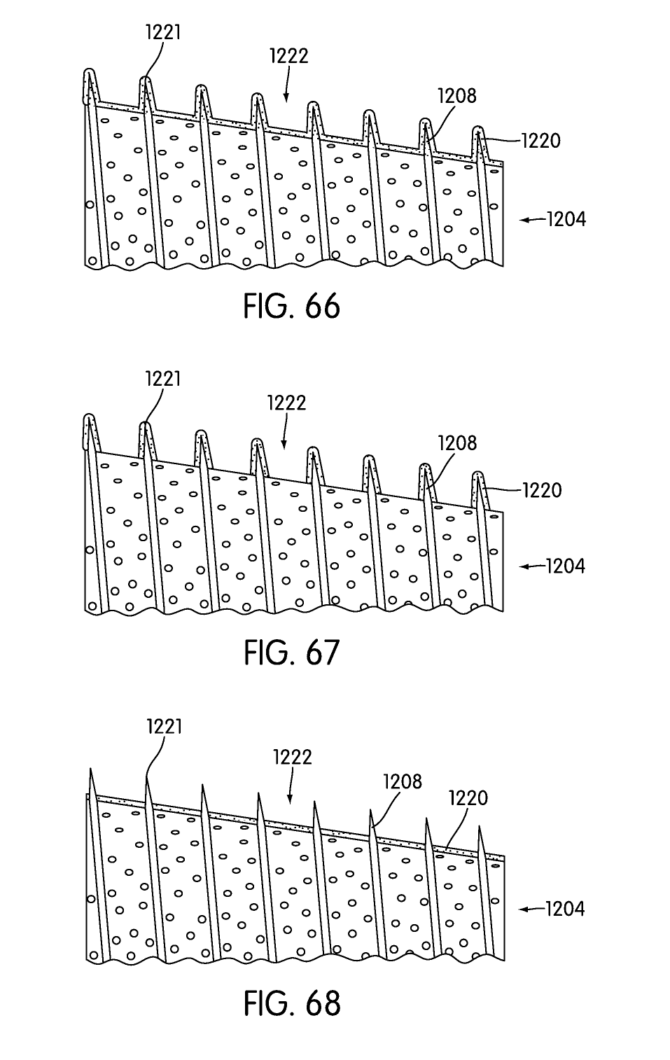

Referring to FIG. 66, a bone growth promoting agent may also be selectively applied to portions of threading 1208. As threading 1208 preferably engages the adjacent vertebrae, a bone growth promoting agent along portions of threading 1208 may facilitate bone growth in the adjacent vertebrae. In this embodiment, bone growth promoting agent 1220 has been applied to threading peaks 1221 of threading 1208 as well as threading valleys 1222 of threading 1208. This coating of the entirety of threading 1208 may be accomplished by dipping threading 1208 in a chemical including bone growth promoting agent 1220. In other embodiments, the coating of threading 1208 may be accomplished using a plasma spray or a similar kind of chemical treatment.

Additionally, it may be desirable in some cases to only coat a portion of threading 1208. Referring to FIG. 67, it may be possible to only apply bone growth promoting agent 1220 to threading peaks 1221 of threading 1208. With this arrangement, threading valleys 1222 may not include bone growth promoting agent 1220. This feature may be accomplished by quickly dipping threading 1208 into a chemical including bone growth promoting agent 1220. By dipping threading 1208 quickly, no time is allowed for the chemical to fill threading valleys 1222. In other embodiments, the coating of threading 1208 may be accomplished using a plasma spray or a similar kind of chemical treatment.

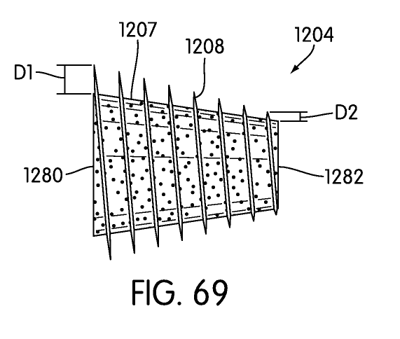

In some cases, only threading valleys 1222 may be coated. Referring to FIG. 68, threading valleys 1222 of threading 1208 may be coated with bone growth promoting agent 1220. This may be accomplished by dipping threading 1208 into a chemical including bone growth promoting agent 1220, and then spinning spinal implant 1204 in a manner that expels bone growth promoting agent 1220 from threading peaks 1221. In other embodiments, the coating of threading 1208 may be accomplished using a plasma spray or a similar kind of chemical treatment.

FIGS. 64-68 are only meant to be illustrative of the various ways a bone growth promoting agent could be selectively applied to spinal implant 1204. In other embodiments, various other portions of spinal implant 1204, including portions of inner surface 1217 and threading 1208, may include bone growth promoting agents. Generally, bone growth promoting agents may be selectively applied throughout any portion of spinal implant 1204, including inner surface 1217 and threading 1208.

Referring to FIG. 69, in some embodiments, spinal implant 1204 may also be a self tapping screw. Self tapping screws are generally any screw that may be inserted without the use of a pilot hole. In this preferred embodiment, spinal implant 1204 may include threading 1208 that is separated a first distance D1 from outer surface 1207 at first end 1280 and that is separated a second distance D2 from outer surface 1207 at second end 1282. First distance D1 is preferably larger than second distance D2. This preferred arrangement allows spinal implant 1204 to be inserted more easily between adjacent vertebrae.

Preferably, a self tapping spinal implant may be configured to promote ingrowth of bone and fuse with the adjacent vertebrae. In some embodiments, a spinal implant including holes and a hollow central core may be implanted between two adjacent vertebrae. Once the spinal implant has been implanted between the vertebrae, growth may occur through the holes into the hollow central core. In a preferred embodiment, the outer and inner surfaces of the spinal implant may be coated with a bone growth promoting agent. In this way, the portions of the bone may fuse together through holes in the spinal implant. Additionally, the spinal implant itself may be fused with the adjacent bone, providing a more stable implant.

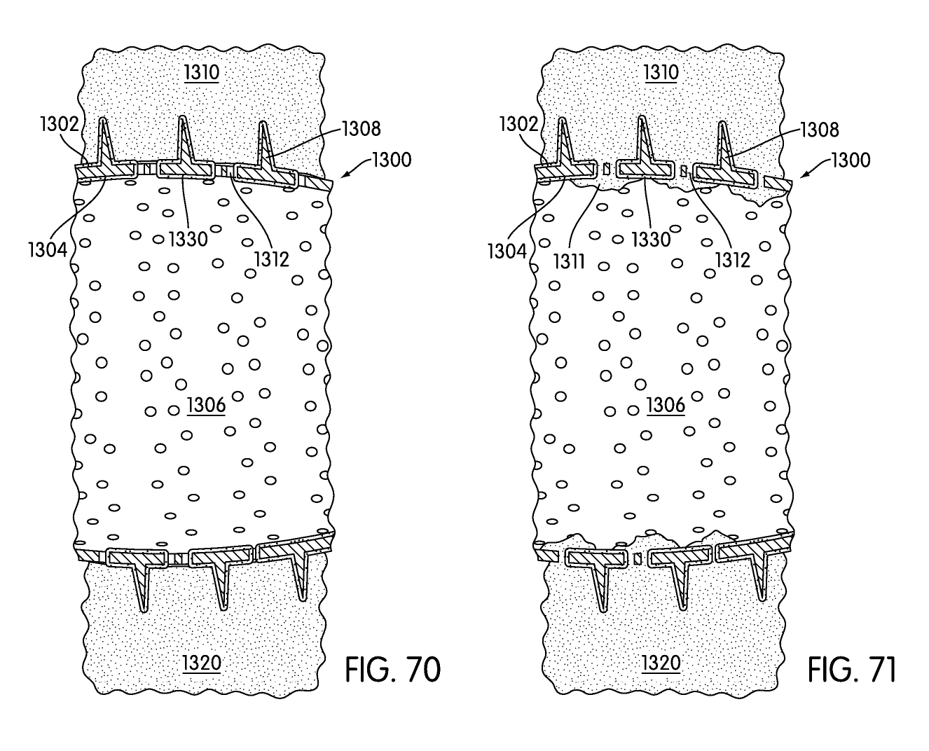

Referring to FIGS. 70-11, the ingrowth of bone from outer surface 1302 through to inner surface 1304 may proceed once spinal implant 1300 has been inserted between first vertebra 1310 and second vertebra 1320. In this embodiment, spinal implant 1300 has a fish or barrel like shape. Generally, threading 1308 may be engaged with vertebrae 1310 and 1320 following the insertion of spinal implant 1300. With time, portions 1311 of first vertebra 1310 and second vertebra 1320 may grow through holes 1312 into hollow central core 1306. In some embodiments, portions 1311 may grow and fill a majority of the space within hollow central core 1306. With this preferred arrangement, spinal implant 1300 may be fused with first vertebra 1310 and second vertebra 1320. In a preferred embodiment, holes 1312 may be used in conjunction with bone growth promoting agent 1330 disposed along inner surface 1304 and outer surface 1302 in order to induce bone growth. Using this configuration, spinal implant 1300 may be partially or fully integrated into vertebrae 1310 and 1320 as they heal. This may allow fusion to occur without the use of bone grafts, bone substitutes, BMP or other similar healing provisions.

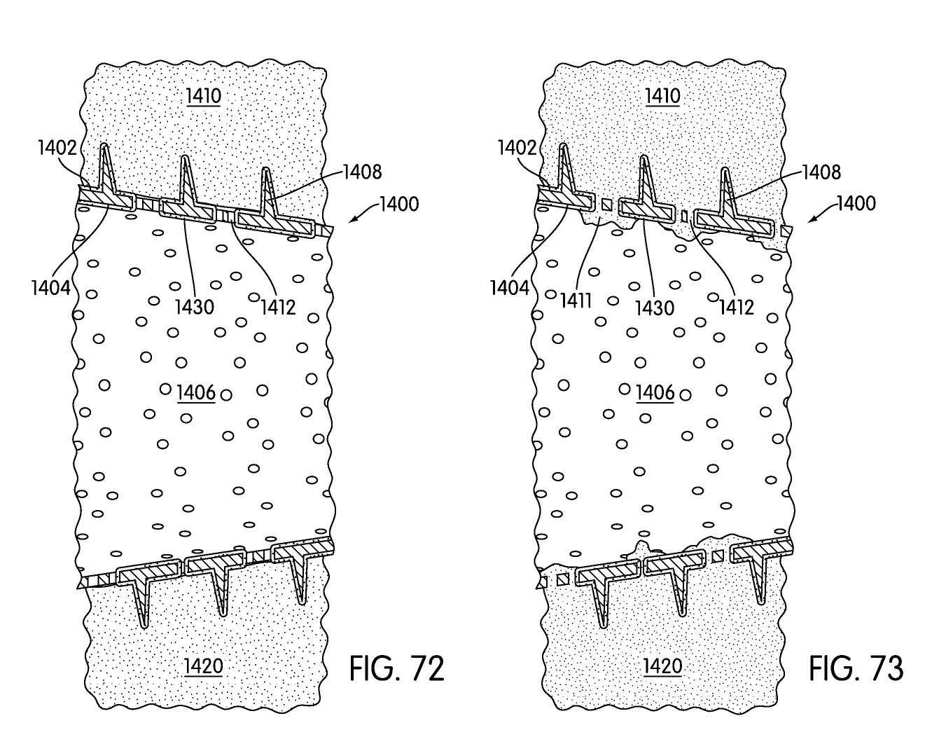

In a similar manner, bone preferably grows through holes associated with the conically shaped embodiment of a spinal implant. Referring to FIGS. 72-73, the ingrowth of bone from outer surface 1402 to inner surface 1404 may proceed once spinal implant 1400 has been inserted between first vertebra 1410 and second vertebra 1420. Generally, threading 1408 may be engaged with vertebrae 1410 and 1420 following the insertion of spinal implant 1400. With time, portions 1411 of first vertebra 1410 and second vertebra 1420 may grow through holes 1412 into hollow central core 1406. In some embodiments, portions 1411 may grow and fill a majority of the space within hollow central core 1406. With this preferred arrangement, spinal implant 1400 may be fused with first vertebra 1410 and second vertebra 1420. In a preferred embodiment, holes 1412 may be used in conjunction with bone growth promoting agent 1430 disposed along inner surface 1404 and outer surface 1402 in order to induce bone growth. Using this configuration, spinal implant 1400 may be partially or fully integrated into vertebrae 1410 and 1420 as they heal. This may allow fusion to occur without the use of bone grafts, bone substitutes, BMP or other similar healing provisions.

In the current embodiments, each spinal implant includes a hollow central core. It should be understood, however, that in other embodiments, each spinal implant could be solid rather than hollow. In these alternative embodiments, each spinal implant may also include holes, including any arrangement for the holes discussed for the previous embodiments.

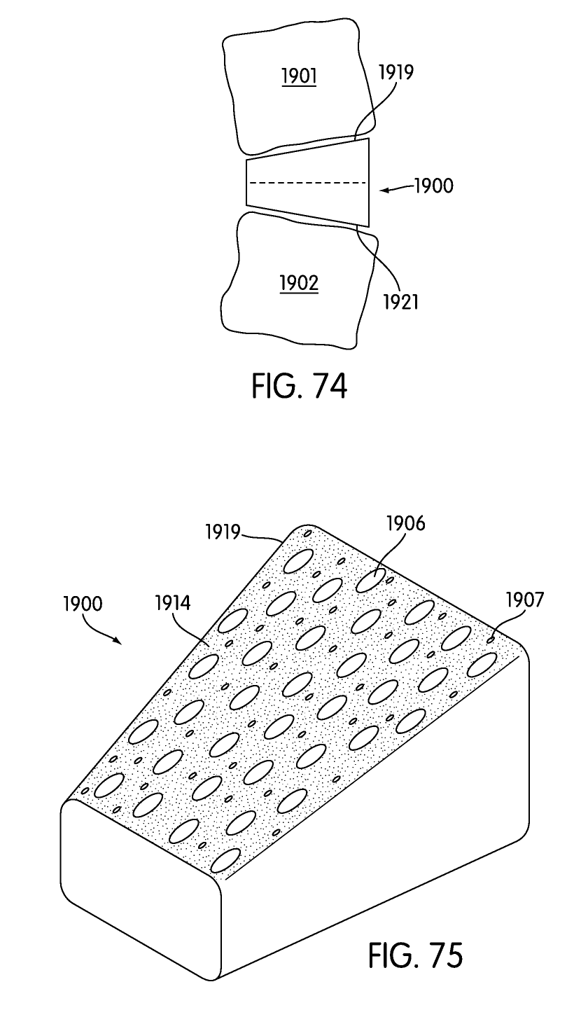

Referring to FIGS. 74-75, an alternative embodiment of a spinal implant is spinal wedge 1900. In a manner similar to the spinal implant discussed in the previous embodiment, spinal wedge 1900 may be disposed between first vertebra 1901 and second vertebra 1902. In some embodiments, a space is made between first vertebra 1901 and second vertebra 1902 prior to the insertion of spinal wedge 1900. During surgery, a surgeon may use a scalpel to cut a window in the outer layer of the intervertebral disc and may then remove the inside portion of the intervertebral disc. In some cases, once a portion of the intervertebral disc is removed, the end plates of vertebrae 1901 and 1902 may be scored to prepare the bone, which preferably initiates a bone healing cascade. Following this, spinal wedge 1900 may be inserted into the space where portions of the intervertebral disc have been removed.

Generally, spinal wedge 1900 may be hollow, with large holes 1906 disposed along upper surface 1919. Additionally, spinal wedge 1900 may include small holes 1907 disposed along upper surface 1919. Preferably, a lower surface 1921 may also include various holes. Generally, large holes 1906 and small holes 1907 may be included to help stimulate bone growth in adjacent vertebrae. In particular, while bone from adjacent vertebrae may grow through large holes 1906 into a hollow central core, small holes 1907 preferably maximize the surface area of spinal wedge 1900 used to induce bone growth. This may allow for healing without the use of bone grafts, bone substitutes, BMP and other similar healing provisions. In other embodiments, spinal wedge 1900 may be solid, rather than hollow, and may or may not include any holes.

In a preferred embodiment, upper surface 1919 may include bone growth promoting agent 1914. With this configuration, bone adjacent to upper surface 1919 may be induced to grow through large holes 1906 of spinal wedge 1900. In some cases, bone may also grow into small holes 1907. Preferably, lower surface 1921 also includes a bone growth promoting agent that may stimulate bone growth as well.

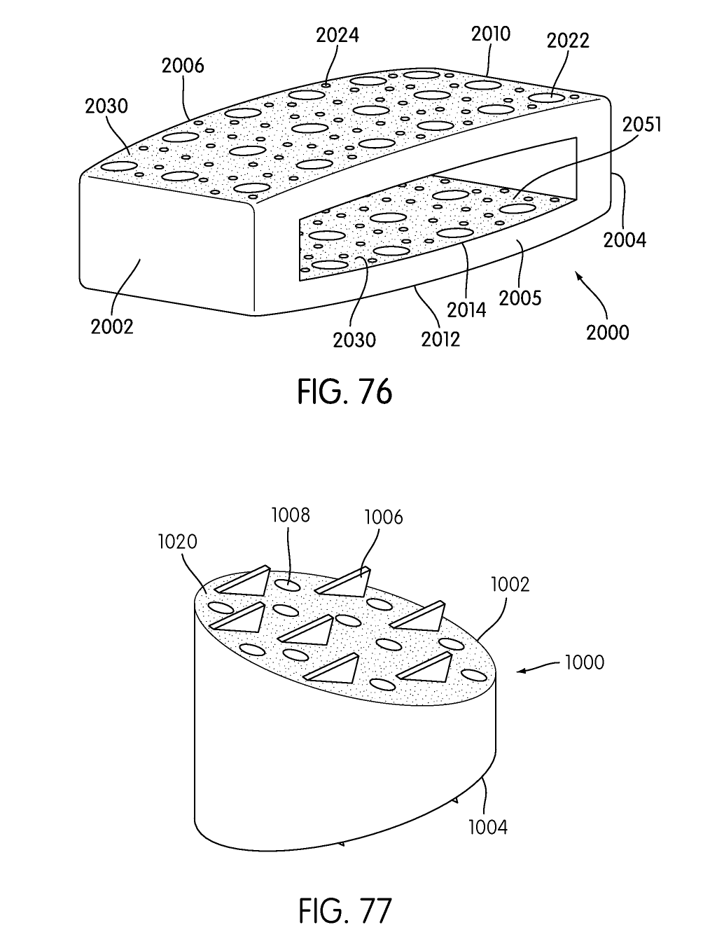

In some embodiments, a spinal implant may be rectangular. FIG. 76 is an exemplary embodiment of spinal plug 2000. Preferably, spinal plug 2000 has a generally rectangular shape, including a rectangular front side 2002 and rear side 2004. Preferably, however, first side 2005 and second side 2006 are generally bowed in the middle, giving spinal plug 2000 a generally bowed shape. This preferred shape may help create lordosis. Generally, the implantation of spinal plug 2000 may proceed in a similar manner to the processes of inserting a spinal wedge, as previously discussed.

Preferably, top side 2010, bottom side 2012, as well as first side 2005 and second side 2006 include provisions for facilitating bone growth. In some embodiments, sides 2005, and 2006 may be associated with large gaps. In particular, first side 2005 may include first large gap 2014 and second side 2006 may include a second large gap (not shown). In some cases, first large gap 2014 and the second large gap may be associated with hollow central core 2051. Additionally, in a preferred embodiment, top side 2010 and bottom side 2012 may include large holes 2022 and small holes 2024 that are both configured to stimulate bone growth. Also, in this preferred embodiment, hollow central core 2051 may include large holes 2022 and small holes 2024 that are both configured to promote bone growth into hollow central core 2051.

Preferably, spinal plug 2000 may also include selectively applied bone growth promoting agent 2030. Generally, bone growth promoting agent 2030 may be disposed on top side 2010 and bottom side 2012, as sides 2010 and 2012 may be configured to contact surfaces of adjacent vertebrae. In some embodiments, bone growth promoting agent 2030 may be disposed inside hollow central core 2051. In other embodiments, bone growth promoting agent 2030 may be disposed in all or some of holes 2022 and 2024.

Referring to FIG. 77, an alternative embodiment of a spinal implant is implantable device 1000. In some embodiments, implantable device 1000 may be cylindrical. In a preferred embodiment, implantable device 1000 may include sloped top side 1002 and sloped bottom side 1004. With this configuration, implantable device 1000 may have a wedge-like shape, decreasing the tendency of implantable device 1000 to shift position.

Preferably, the implantation of implantable device 1000 may proceed in a similar manner to the processes of inserting a spinal plug and a spinal wedge. In some embodiments, implantable device 1000 may include teeth 1006. With this arrangement, teeth 1006 preferably help decrease the tendency of implantable device 1000 to slip. Additionally, implantable device 1000 may include holes 1008. In a preferred embodiment, holes 1008 may facilitate bone growth into implantable device 1000.

In a preferred embodiment, implantable device 1000 may also include selectively applied bone growth promoting agent 1020. In some embodiments, bone growth promoting agent 1020 may be selectively applied to top side 1002 and bottom side 1004. In some embodiments, bone growth promoting agent 1020 may also be selectively applied to teeth 1006. This configuration preferably stimulates bone growth of the adjacent vertebrae into teeth 1006, on top side 1002 and bottom side 1004, once implantable device 1000 has been inserted into a spine.

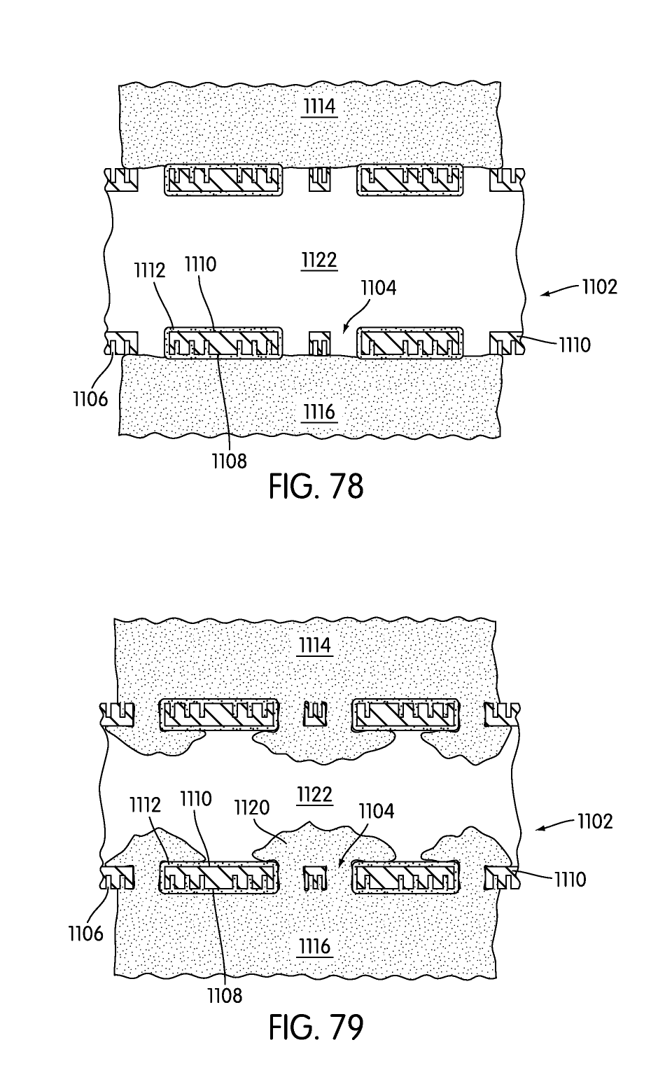

As with previous embodiments, spinal wedge 1900, spinal plug 2000 and implantable device 1000 may be configured with holes that facilitate new bone growth. FIGS. 78-79 are cross sectional views of a preferred embodiment of spinal implant 1102, including sides 1100 and hollow central core 1122. Spinal implant 1102 could be a spinal plug, a spinal wedge or an implantable device. For the purposes of illustration, spinal implant 1102 is shown as rectangular, but it should be understood that the general principles discussed here may apply to other similar spinal implants.

In some embodiments, spinal implant 1102 includes large holes 1104 and small holes 1106. In this embodiment, large holes 1104 are configured to penetrate through spinal implant 1102, from outer surface 1108 to inner surface 1110. Also, small holes 1106 may only disposed on outer surface 1108. In other words, small holes 1106 do not penetrate through to hollow central core 1122. In other cases, small holes 1106 may penetrate through spinal implant 1102, while large holes 1104 are only disposed on outer surface 1108 and do not fully penetrate through spinal implant 1102. In still other embodiments, small holes 1106 and large holes 1104 may be surface features that do not penetrate completely through spinal implant 1102. Finally, in a preferred embodiment, both small holes 1106 and large holes 1104 penetrate through spinal implant 1102. Any combination of the hole configurations may also be used. By changing the depths of holes 1104 and 1106, the fusion inducing properties of spinal implant 1102 may be varied.

Preferably, in some embodiments, spinal implant 1102 may include bone growth promoting agent 1112. In some embodiments, bone growth promoting agent 1112 may be selectively applied to various portions of spinal implant 1102. In a preferred embodiment, bone growth promoting agent 1112 may be selectively applied to outer surface 1108 and inner surface 1110.

As with the previous embodiments, the ingrowth of bone from outer surface 1108 to inner surface 1110 may proceed once spinal implant 1102 has been inserted between first vertebra 1114 and second vertebra 1116. With time, portions 1120 of first vertebra 1114 and second vertebra 1116 may grow into or through large holes 1104 into hollow central core 1122 and into or through small holes 1106. In some embodiments, portions 1120 may grow to fill a majority of the space within hollow central core 1122. In this way, spinal implant 1102 may be fused with first vertebra 1114 and second vertebra 1116. In a preferred embodiment, holes 1104 and 1106 may be used in conjunction with bone growth promoting agent 1112 disposed along inner surface 1110 and outer surface 1108 in order to induce bone growth. Additionally, in some embodiments, bone growth promoting agent 1112 may be selectively applied to any portion of spinal implant 1102, including holes 1104 and 1106. With this arrangement, spinal implant 1102 may be partially or fully integrated into vertebrae 1114 and 1116 as they heal.

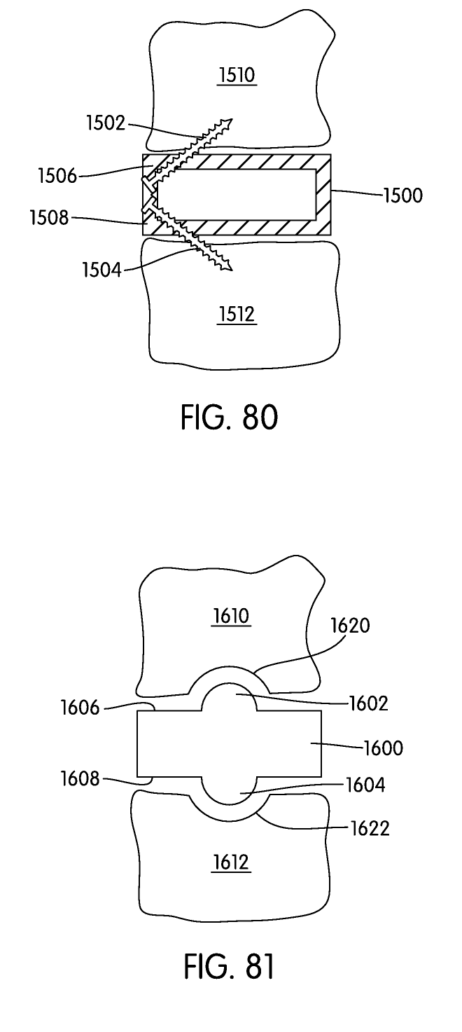

Referring to FIGS. 80-83, some embodiments may include additional provisions for securing the spinal implants in place between vertebrae. For the purposes of clarity, the following embodiments are shown as a generic rectangular spinal implant. However, it should be understood that many of these additional provisions may be used with multiple types of spinal implants, including, spinal implants, spinal wedges, spinal plugs, and implantable devices, as well as other spinal implants. Additionally, each of the following provisions may be used in conjunction with a selectively applied bone growth promoting agent.

In some cases, additional screws may be used with spinal implant 1500, as seen in FIG. 80. The following embodiment is one example of a spinal implant that incorporates additional screws. Further examples can be found in U.S. Pat. No. 7,018,412, the entirety of which is incorporated here by reference. In this embodiment, first screw 1502 and second screw 1504 may be inserted through upper corner 1506 and lower corner 1508 of spinal implant 1500, respectively. Furthermore, first screw 1502 may be inserted into first vertebra 1510 and second screw 1504 may be inserted into second vertebra 1512. Preferably, spinal implant 1500 includes provisions for receiving screws 1502 and 1504 at upper corner 1506 and lower corner 1508, respectively. Using this configuration, spinal implant 1500 may be secured firmly into place between vertebrae 1510 and 1512. In other embodiments, a spinal implant may be secured to vertebrae 1510 and 1512 using more than two screws.

In another embodiment, a spinal implant may include provisions for locking into place between adjacent vertebrae. Examples of such provisions can be found in U.S. Pat. Nos. 6,332,895; 6,045,580; 6,547,823; and 7,018,412, the entirety of which are incorporated by reference. In FIG. 81, spinal implant 1600 includes first central protrusion 1602 disposed on upper side 1606 and second central protrusion 1604 on lower side 1608. Preferably, first vertebra 1610 includes first recess 1620 that is configured to receive first central protrusion 1602. Likewise, second vertebra 1612 may include second recess 1622 that is configured to receive second central protrusion 1604. Using this configuration, first central protrusion 1602 and second central protrusion 1604 may prevent spinal implant 1600 front slipping horizontally with respect to vertebrae 1610 and 1612.

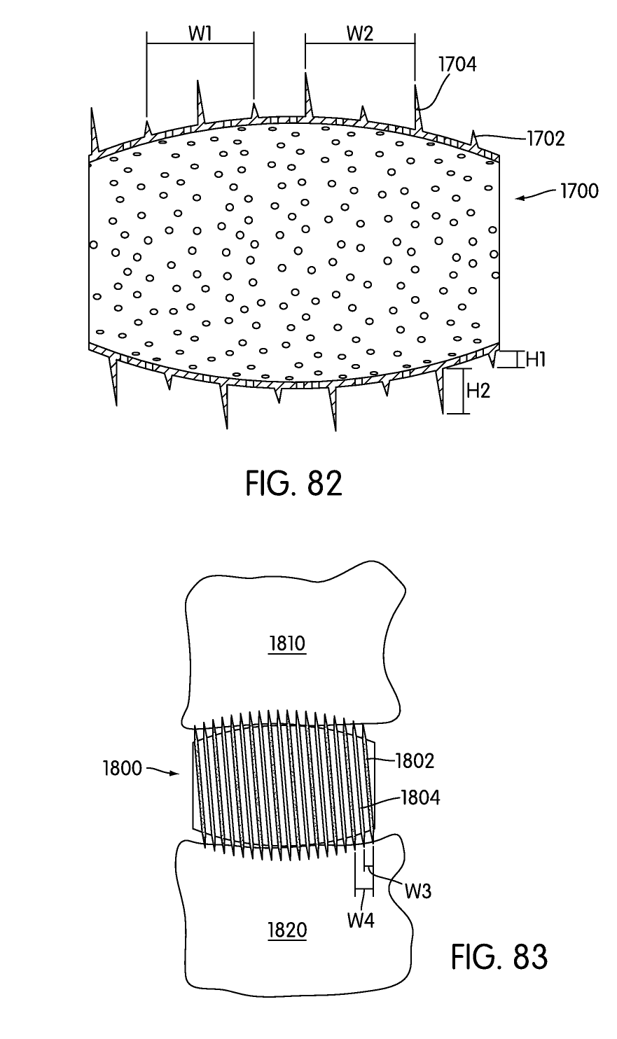

In other embodiments, various types of threading may be used with spinal implants. FIG. 82 is a cross sectional view of a preferred embodiment of spinal implant 1700. In this embodiment, spinal implant 1700 may be similar to spinal implant 504, seen in FIG. 59. Preferably, spinal implant 1700 may include a double helix threading, as opposed to the traditional threading seen in the previous embodiments. In this embodiment, spinal implant 1700 may be associated with first threading 1702 and second threading 1704. First threading 1702 may be wound around spinal implant 1700 with first lead width W1. Likewise, second threading 1704 may be wound around spinal implant 1700 with second lead width W2. Furthermore, first threading 1702 is preferably associated with a height H1 that is smaller than a height H2 that may be associated with second threading 1704. In other embodiments, first threading 1702 and second threading 1704 may be the same size. Using this preferred configuration, spinal implant 1700 may more easily penetrate between adjacent vertebrae.

FIG. 83 is a side view of a preferred embodiment of spinal implant 1800. In this embodiment, spinal implant 1800 may include first threading 1802 and second threading 1804. In other words, spinal implant 1800 may be double threaded. In particular, lead width W3 is twice the pitch width W4. With this double threaded configuration, spinal implant 1800 may be inserted between first vertebra 1810 and second vertebra 1820 more quickly than a single threaded screw. This may be useful in reducing rotation, migration or pull-out of spinal implant 1800.

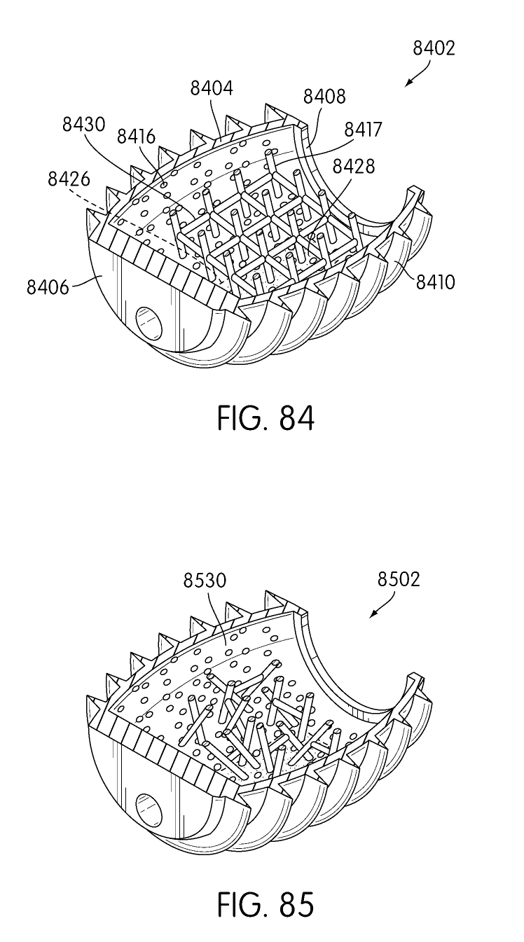

FIG. 84 is an alternative embodiment of a spinal implant. As disclosed above, some spinal implant embodiments may be hollow, like the one shown in FIG. 64 above, and other spinal implant embodiments may be solid. It is also possible to create a spinal implant that includes a lattice or frame structure. The lattice or frame structure can be used to provide additional strength to the spinal implant and also to provide open or interstitial spaces for bone penetrating the outer surface of the spinal implant. An example of a latticed spinal implant is shown in FIG. 84.

Referring to FIG. 84, latticed spinal implant 8402 includes outer shell 8404, first axial end portion 8406, and second axial end portion 8408. Outer shell 8404 generally extends axially between first axial end 8406 and second axial end 8408. In some embodiments, outer shell 8404 may include threading 8410. As disclosed above, threading 8410 may assist in implanting and securing latticed spinal implant 8402 between two vertebrae. For illustrative purposes, FIG. 84 only includes the lower half of latticed spinal implant 8402; however latticed spinal implant 8402 also comprises a second upper half not shown here.

Latticed spinal implant 8402 preferably includes inner surface 8417. Preferably, holes 8416 are also disposed along inner surface 8417. As disclosed above, any number, configuration, arrangement, size and/or depth of holes 8416 may be disposed on spinal implant 8402. In the embodiment shown in FIG. 84, holes 8416 are shown to generally visibly penetrate through latticed spinal implant 8402. In other embodiments, holes may not penetrate through the implant or the holes may be so small that they are not visible in FIG. 84.

In the embodiment shown in FIG. 84, latticed spinal implant 8402 includes lattice structure 8430. Lattice structure 8430 can include a regular or irregular system of links or struts. In the embodiment shown in FIG. 84, lattice structure 8430 includes a regular series of links that are connected to one another at roughly 90 degree angles. This arrangement is similar to a series of cubes, formed by links, that are sequentially attached to one another. Lattice structure 8430 is preferably attached to latticed spinal implant 8402. However, in some embodiments, lattice structure 8430 is not attached to latticed spinal implant 8402 and can move with respect to latticed spinal implant 8402.

Preferably, latticed spinal implant 8402 includes some kind of bone growth promoting agent that encourages bone growth to and through latticed spinal implant 8402. In the embodiment shown in FIG. 84, inner surface 8417 may include a first portion 8426. In a preferred embodiment, first portion 8426 of inner surface 8417 may include bone growth promoting agent 8428. With this configuration, bone may be induced to grow through holes 8416 disposed in shell 8404. It should be kept in mind that first portion 8426 is merely exemplary of the size, shape, design and location of bone growth promoting agent 8428. In other embodiments, bone growth promoting agent 8428 may be applied to any number of areas, and in any number of patterns, configurations and sizes disclosed above.

Preferably, lattice structure 8430 may be configured to cooperate with bone growth promoting agent 8428. This may be done to encourage penetrating bone growth to fuse with lattice structure 8430 and/or incorporate lattice structure 8430 into the final bone matrix. To encourage this fusion to lattice structure 8430, bone growth promoting agent 8428 may be applied to the entire lattice structure 8430 or to selected portions of lattice structure 8430. Any of the patterns, configurations or systems of bone growth promoting agents disclosed above may be applied to lattice structure 8430.

FIG. 85 is an alternative embodiment showing an irregular lattice structure 8530 disposed in second latticed spinal implant 8502. Irregular lattice structure 8530 may include a random or nearly random connected system of links. These links are generally not connected to one another at regular intervals or at regular angles. Also, the links in this embodiment may be of unequal lengths. All of the features related to the application of bone growth promoting agents disclosed in connection with the previous embodiment may be applied to this embodiment, shown in FIG. 85 as well.

While the examples showing the lattice structures are applied to the fish-shaped spinal implants, it should be kept in mind that any implant or device may include a lattice structure. These geometric lattice structures can be regular, irregular or any combination thereof.

In some embodiments, a fusion system may include additional provisions for facilitating the fusion of two adjacent bones. In some cases, the fusion system may include one or more bone staples. In a preferred embodiment, the one or more bone staples may be used simultaneously with a fusion device in order to facilitate increased bony fusion between two adjacent bones and to provide increased structural support.

In a manner similar to the previous embodiments, bone staples may include provisions for stimulating the growth of adjacent bone. In some cases, a bone growth promoting agent may be selectively applied to one or more bone staples to facilitate bone growth along one or more portions of a bone staple. In a preferred embodiment, a bone growth promoting agent may be selectively applied to a portion of the bone staple that is adjacent to one or more vertebrae.

As previously mentioned, bone growth promoting agents can be selectively applied in any shape and/or pattern. Additionally, in some cases, a combination of different bone growth promoting agents may be used. Also, bone growth promoting agents may be used simultaneously with surface treatments of bone staples, in a manner similar to the use of surface treatments that were previously discussed for rods. It should be understood that each of the applications of one or more bone growth promoting agents or bone growth facilitating features that have been discussed for rods may be similarly applied to bone staples.

In some embodiments, the bone staples may also include holes. In some cases, macro holes may be used. In other cases, micro holes may be used. In a preferred embodiment, macro and micro holes may be used in combination with a selectively applied bone growth promoting agent.

Generally, bone staples may be applied to adjacent vertebrae by associating one end of the bone staple with one vertebra and a second end of the bone staple with another vertebra. In some cases, the bone staple may be inserted using various tools such as hammers, drills, or other devices. In other cases, the bone staple may be inserted using a pneumatic device or a spring based device that is configured to forcefully insert a large staple into bone.

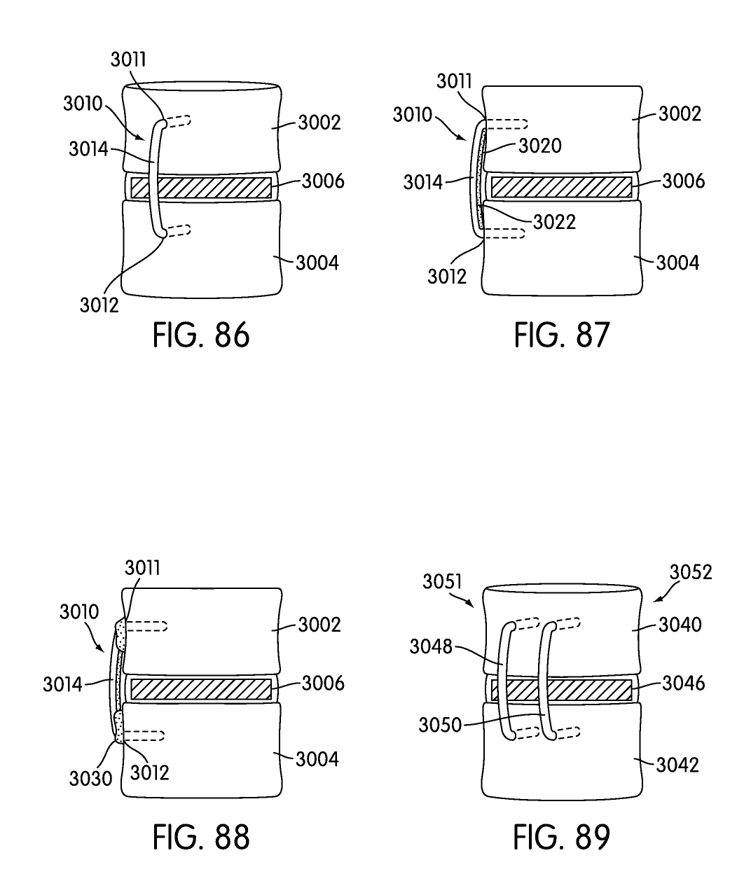

Referring to FIG. 86, spinal fusion device 3006 has been inserted between first vertebra 3002 and second vertebra 3004. Generally, spinal fusion device 3006 may be any type of fusion device that has been previously disclosed or that is known in the art. In some cases, first vertebra 3002 may be the L4 lumbar vertebra and second vertebra 3004 may be the L5 lumbar vertebra. In other cases, vertebrae 3002 and 3004 could be any type of vertebrae, including vertebrae from the lumbar, thoracic, cervical or sacral regions of the spine.

In some embodiments, bone staple 3010 may be inserted into vertebrae 3002 and 3004. In particular, first end 3011 of bone staple 3010 may be inserted into first vertebra 3002 and second end 3012 of bone staple 3010 may be inserted into second vertebra 3004. With this arrangement, body portion 3014 of bone staple 3010 may be disposed between vertebrae 3002 and 3004. This configuration may provide increased structural stability for vertebrae 3002 and 3004.

Referring to FIG. 87, in some embodiments, a bone growth promoting agent may be selectively applied to a portion of bone staple 3010. In the current embodiment, bone growth promoting agent 3020 has been selectively applied to inner surface 3022 of body portion 3014. Preferably, bone growth promoting agent 3020 has also been applied to ends 3011 and 3012 of bone staple 3010, prior to the insertion of bone staple 3010. In other embodiments, bone growth promoting agent 3020 could also be applied to additional portions of bone staple 3010 as well.

FIG. 88 is a preferred embodiment of bone staple 3010 including bone growth. In this embodiment, bone growth 3030 has occurred at first end 3011 and second end 3012. Additionally, some of body portion 3014 may include bone growth. In other embodiments, bone growth may occur over all of bone staple 3010.

Preferably, this new bony fusion facilitates the fusion of vertebrae 3002 and 3004 to spinal fusion device 3006 and to one another. In some cases, this preferred arrangement creates a rebar effect, reinforcing the strength of the connection between adjacent vertebrae. This arrangement also helps to incorporate bone staple 3010 into the bone or bones.

Although the current embodiment includes a single bone staple, it should be understood that in other embodiments, any number of bone staples may be used. In some cases, two bone staples may be used. In other cases, more than two bone staples may be used. Additionally, the shape, size, length, thickness as well as other characteristics of a bone staple may be varied.

FIG. 89 is a preferred embodiment of first vertebra 3040 and second vertebra 3042 that are fixed together using spinal fusion device 3046 as well as first bone staple 3048 and second bone staple 3050. In this embodiment, first bone staple 3048 may be associated with a first side 3051 of vertebrae 3040 and 3042. Additionally, second bone staple 3050 may be associated with second side 3052 of vertebrae 3040 and 3042. This arrangement may help facilitate fusion of vertebrae 3040 and 3042 along both sides 3051 and 3052. As with the previous embodiments, one or more bone growth promoting agents may be selectively applied to staples 3048 and 3050 to facilitate bony fusion.

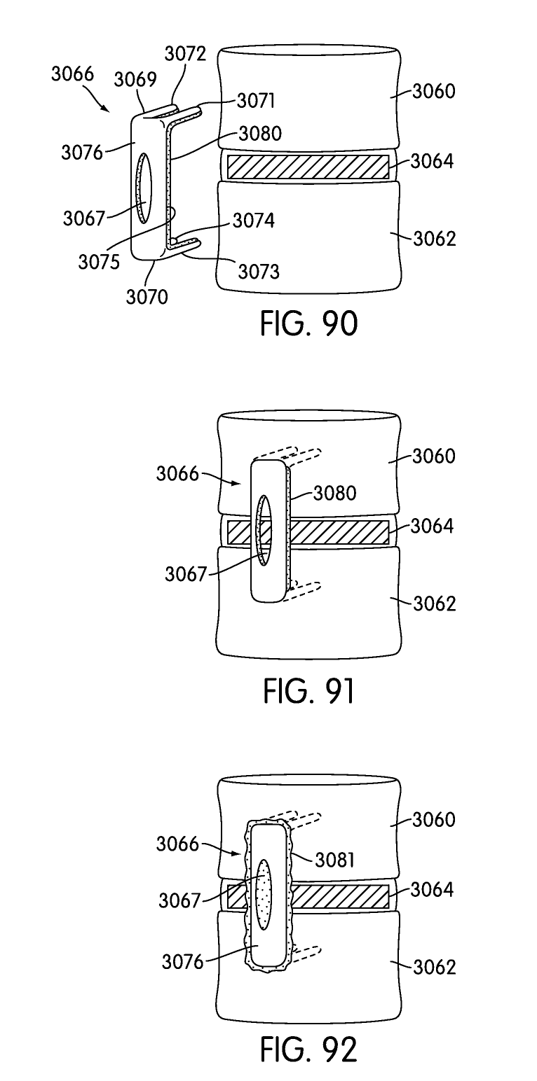

FIGS. 90-92 are intended to illustrate a preferred embodiment of a bone staple with a large width. Referring to FIG. 90, first vertebra 3060 and second vertebra 3062 may be fixedly attached using spinal fusion device 3064 as well as wide bone staple 3066. In this embodiment, wide bone staple 3066 includes provisions for inserting into vertebrae 3060 and 3062. In particular, wide bone staple 3066 includes first inserting portion 3071 and second inserting portion 3072 at first end 3069. Additionally, wide bone staple 3066 may include third inserting portion 3073 and fourth inserting portion 3074 at second end 3070. Using inserting portions 3071-3074, wide bone staple 3066 may be attached to vertebrae 3060 and 3062, as seen in FIG. 91.

Referring to FIG. 90, a bone growth promoting agent may be selectively applied to one or more portions of wide bone staple 3066. In this embodiment, bone growth promoting agent 3080 is preferably applied to inserting portions 3071-3074 as well as inner surface 3075 of body portion 3076. Preferably, using this arrangement, new bone growth may 3081 occur along body portion 3076 to further fuse vertebrae 3060 and 3062 together, as seen in FIG. 92.

Preferably, wide bone staple 3066 includes provisions that allow a surgeon to monitor new bone growth along bone staple 3066, between vertebrae 3060 and 3062. Typically, new bone growth may be observed through x-rays. However, in some cases, metallic materials may prohibit the observation of new bone growth using x-rays. In some cases, a wide bone staple may include one or more holes that allow a surgeon to view new bone growth using x-rays. In a preferred embodiment, a wide bone staple may include a single large hole.

In this embodiment, wide bone staple 3066 may include large hole 3067 that is disposed within body portion 3076. As seen in FIGS. 91 and 92, some portions of spinal fusion device 3064, as well as potions of vertebrae 3060 and 3062 may be visible through large hole 3067. As fusion occurs between vertebrae 3060 and 3062, some portions of new bone growth 3081 may be visible through large hole 3067. This arrangement allows new bone growth to be observed using x-rays so that a surgeon can track the progress of the fusion of vertebrae 3060 and 3062.

While various embodiments of the invention have been described, the description is intended to be exemplary, rather than limiting and it will be apparent to those of ordinary skill in the art that many more embodiments and implementations are possible that are within the scope of the invention. Accordingly, the invention is not to be restricted except in light of the attached claims and their equivalents. Also, various modifications and changes may be made within the scope of the attached claims.

We claim:

1. A spinal fusion device, comprising:
a spinal implant configured for insertion between two vertebrae;
the spinal implant including a first portion and a second portion along an outer surface;
a bone growth promoting agent; and
wherein the bone growth promoting agent is selectively applied to the first portion of the outer surface.

2. The spinal fusion device according to claim 1, wherein the bone growth promoting agent is selectively applied to an inner surface of the spinal implant.

3. The spinal fusion device according to claim 2, wherein the spinal implant includes a plurality of holes.

4. The spinal fusion device according to claim 3, wherein the plurality of holes are disposed on an outer surface of the spinal implant.

5. The spinal fusion device according to claim 3, wherein the plurality of holes includes small holes and large holes.

6. The spinal fusion device according to claim 2, wherein the bone growth promoting agent is selectively applied to at least one of the plurality of holes.

7. The spinal fusion device according to claim 1, wherein the spinal implant has a solid portion.

8. The spinal fusion device according to claim 1, wherein the spinal implant has a hollow portion.

9. The spinal fusion device according to claim 1, wherein the spinal implant has a latticed portion.

10. A spinal fusion device, comprising:
a spinal implant configured for insertion between two vertebrae;
the spinal implant including threading;
the threading including threading peaks and threading valleys;
a bone growth promoting agent; and
wherein the bone growth promoting agent is selectively applied to the threading peaks.

11. The spinal fusion device according to claim 10, wherein the threading peaks include an upper portion, a middle portion and a lower portion.

12. The spinal fusion device according to claim 11, wherein the bone growth promoting agent is selectively applied to a member of the group consisting essentially of the upper portion, the lower portion, the middle portion and the threading valleys.

13. The spinal fusion device according to claim 10, wherein the spinal implant includes a plurality of holes.

14. The spinal fusion device according to claim 13, wherein at least one of the plurality of holes penetrates from an outer surface of the spinal implant to an inner surface associated with a hollow central core.

15. The spinal fusion device according to claim 14, wherein at least one of the plurality of holes has a bottom.

16. A spinal fusion device, comprising:
a spinal plug configured for insertion between two vertebrae;
the spinal plug including a first portion and a second portion along an outer surface;
a bone growth promoting agent; and
wherein the bone growth promoting agent is selectively applied to the first portion of the outer surface.

17. The spinal fusion device according to claim 16, wherein the bone growth promoting agent is selectively applied to a portion of an inner surface of the spinal plug.

18. The spinal fusion device according to claim 16, wherein the spinal plug has a solid portion.

19. The spinal fusion device according to claim 16, wherein the spinal plug has a hollow portion.

20. The spinal fusion device according to claim 16, wherein the spinal plug has a latticed portion.

21. The spinal fusion device according to claim 16, wherein the spinal plug includes a plurality of holes.

22. The spinal fusion device according to claim 21, wherein the bone growth promoting agent is selectively applied to at least one of the holes.

23. The spinal fusion device according to claim 22, wherein the plurality of holes are disposed on a top side and a bottom side of the spinal plug.

24. The spinal fusion device according to claim 23, wherein the plurality of holes includes small holes and large holes.

25. A spinal fusion device, comprising:
a spinal wedge configured for insertion between two vertebrae;
the spinal wedge including a first portion and a second portion along an outer surface;
a bone growth promoting agent; and
wherein the bone growth promoting agent is selectively applied to the first portion of the outer surface.

26. The spinal fusion device according to claim 25, wherein the spinal wedge includes a hollow portion.

27. The spinal fusion device according to claim 25, wherein the spinal wedge includes a solid portion.

28. The spinal fusion device according to claim 25, wherein the spinal wedge includes a latticed portion.

29. The spinal fusion device according to claim 25, wherein the implantable device includes a plurality of holes.

30. The spinal fusion device according to claim 29, wherein the plurality of holes are disposed on a top side and a bottom side of the spinal wedge.

31. The spinal fusion device according to claim 29, wherein the plurality of holes includes small holes and large holes.

32. The spinal fusion device according to claim 29, wherein the bone growth promoting agent is selectively applied to at least one of the plurality of holes.

33. A spinal fusion device, comprising:
an implantable device configured for insertion between two vertebrae;
the implantable device including a first portion and a second portion along an outer surface;
a bone growth promoting agent; and
wherein the bone growth promoting agent is selectively applied to the first portion of the outer surface.

34. The spinal fusion device according to claim 33, wherein the implantable device includes teeth.

35. The spinal fusion device according to claim 33, wherein the implantable device includes a sloped top side.

36. The spinal fusion device according to claim 33, wherein the implantable device includes a sloped bottom side.

37. The spinal fusion device according to claim 36, wherein the implantable device includes a plurality of holes.

38. The spinal fusion device according to claim 37, wherein the plurality of holes are disposed on the sloped top side and the sloped bottom side.

39. The spinal fusion device according to claim 36, wherein the plurality of holes includes small holes and large holes.

40. The spinal fusion device according to claim 37, wherein the bone growth promoting agent is selectively applied to at least one of the plurality of holes.

41. A spinal fusion device, comprising:
an implantable device configured for insertion between two vertebrae;
the implantable device including a first portion and a second portion;
a bone growth promoting agent;
a lattice structure disposed within the implantable device; and
wherein the bone growth promoting agent is selectively applied to the first portion.

42. The spinal fusion device according to claim 41, wherein the first portion includes a portion of a shell of the implantable device.

43. The spinal fusion device according to claim 42, wherein the first portion also includes a portion of the lattice structure, wherein the bone growth promoting agent applied to both the shell and the lattice structure encourages bone growth into the lattice structure and bone integration with the lattice structure.

44. The spinal fusion device according to claim 41, wherein the first portion includes a portion of the lattice structure.

45. The spinal fusion device according to claim 41, wherein the lattice structure is removable from the spinal fusion device.

46. A spinal fusion device, comprising:
an implantable device including a surface associated with a vertebra;
the surface including a hole; and
wherein a bone growth promoting agent is selectively applied to a portion of the hole.

47. The spinal fusion device according to claim 46, wherein the hole extends through the surface.

48. The spinal fusion device according to claim 46, wherein the hole includes a bottom.

49. The spinal fusion device according to claim 46, wherein the hole is microscopic.

50. The spinal fusion device according to claim 46, wherein the hole is macroscopic.

51. A bone fusion device, comprising:
a body portion;
a first inserting portion extending from the body portion;
a second inserting portion extending from the body portion;
wherein the first inserting portion engages a first bone and wherein the second inserting portion engages a second bone; and
wherein a bone growth promoting agent is selectively applied to a portion of the bone fusion device.

52. The bone fusion device according to claim 51, wherein the bone fusion device includes at least one hole, and wherein the hole is microscopic.

53. The bone fusion device according to claim 51, wherein the bone fusion device includes at least one hole, and wherein the hole is macroscopic.

54. The bone fusion device according to claim 51, wherein the first bone is a vertebrae and wherein the second bone is an adjacent vertebrae.

55. The bone fusion device according to claim 51, wherein the bone fusion device includes at least two inserting portions.

56. The bone fusion device according to claim 55, wherein the bone fusion device includes at least four inserting portions.
